(19) United States
(12) Patent Application Publication
(10) Pub. No.: US 2026/0035457 A1
(43) Pub. Date: Feb. 5, 2026

(54) ANTI-NOTCH3 ANTIBODY

(71) Applicant: AVEO Pharmaceuticals, Inc., Boston, MA (US)

(72) Inventors: Heidi Okamura, Brookline, MA (US); William M. Winston, Jr., Marlborough, MA (US); Laura Poling, Boston, MA (US); Alisa C. Bell, Bedford, MA (US)

(21) Appl. No.: 19/216,354

(22) Filed: May 22, 2025

Related U.S. Application Data
(63) Continuation of application No. 17/450,282, filed on Oct. 7, 2021, now Pat. No. 12,338,282.
Application No. 17/450,282 is a continuation of application No. 16/097,489, now Pat. No. 11,142,573.
(60) Provisional application No. 62/329,729, filed on Apr. 29, 2016.
(60) Provisional application No. 62/460,371, filed on Feb. 17, 2017.

(51) Int. Cl. C07K 16/28; A61K 39/00; A61K 39/395; A61P 35/00; C07K 14/71; C07K 16/30; C12N 5/10; C12N 5/12; C12N 15/63

(52) U.S. Cl. CPC C07K 16/28; A61K 39/3955; A61K 39/39558; A61P 35/00; C07K 16/2863; C07K 16/30; C12N 5/10; C12N 5/12; C12N 15/63; A61K 2039/505; C07K 14/71; C07K 2317/21; C07K 2317/33; C07K 2317/56; C07K 2317/565; C07K 2317/567; C07K 2317/76; C07K 2317/92

(57) ABSTRACT

Monoclonal antibodies that bind and inhibit activation of human Notch3 are disclosed. The antibodies can be used to treat cell proliferative diseases and disorders, including certain forms of cancer, associated with activation and/or overexpression of Notch3.

### CROSS-REFERENCE TO RELATED APPLICATIONS

This application is a continuation of U.S. patent application Ser. No. 17/450,282 filed Oct. 7, 2021, which is a continuation of U.S. patent application Ser. No. 16/097,489 filed Oct. 29, 2018, which is a U.S. national stage application under 35 U.S.C. § 371 of International Application No. PCT/US2017/030156 filed Apr. 28, 2017, which claims the benefit of and priority to U.S. Provisional Patent Application No. 62/460,371 filed Feb. 17, 2017, and U.S. Provisional Application No. 62/329,729, filed Apr. 29, 2016, the contents of each of which are incorporated by reference herein in their entirety.

### FIELD OF THE INVENTION

The field of the invention is molecular biology, specifically immunology and antibodies that bind human Notch3.

### BACKGROUND

Notch pathway signaling is involved in numerous cellular processes, including cell fate determination, differentiation, proliferation, apoptosis, migration and angiogenesis. In mammals, there are four Notch proteins (sometimes called “Notch receptors”), designated Notch1-Notch4. All four Notch proteins have a similar domain structure, which includes an extracellular domain, a negative regulatory (NRR) domain, a single-pass transmembrane domain, and an intracellular domain. The extracellular domain contains a series of EGF-like repeats that are involved in ligand binding. During maturation, the Notch polypeptide is cleaved by a furin-like protease. This cleavage divides the Notch protein into two subunits that are held together by the NRR. In the absence of ligand binding, the NRR domain functions to keep the Notch protein in a protease-resistant conformation. The intracellular domain is a transcription factor called Notch intracellular domain (NICD), which is released upon proteolytic cleavage by gamma secretase, in response to binding of the Notch protein by a ligand. In mammals, the Notch ligands are Delta-like (e.g., DLL1 and DLL4) and Jagged (also referred to as Jag, e.g., Jag1 and Jag2). When the NICD is released, it travels to the nucleus, where it activates transcription of the Notch-responsive genes, HES1, HES5, NRARP, Deltex1 and c-MYC. For reviews of Notch-related biology, see, e.g., Bray, 2006, NATURE REVIEWS 7:678-689; Kopan et al., 2009, CELL 137:216-233.

While Notch proteins play crucial roles in normal development, dysregulation of the Notch proteins is associated with various types of cancer, including T-cell acute lymphatic leukemia/lymphoma (T-All), breast cancer, colon cancer, ovarian cancer and lung cancer. See, e.g., Miele et al., 2006, CURRENT CANCER DRUG TARGETS 6:313-323. Accordingly, one therapeutic approach for the treatment of cancer is inhibition of Notch pathway signaling. Inhibition of Notch pathway signaling has been achieved using monoclonal antibodies (Wu et al., 2010, NATURE 464:1052-1057; Aste-Amézaga et al., 2010, PLOS ONE 5:1-13 e9094). Further, dysregulation of Notch may be associated with development of pulmonary arterial hypertension (PAH). Accordingly, a therapeutic approach to treating PAH may involve inhibition of the Notch pathway signaling. (Li et al., 2009, Nature Medicine, 15:1289-1299; Jalali et al., 2012, PLOS ONE 7:e46808; Yu et al., 2013, Am. J. Respir. Cell Mol. Biol., 48:647-654; Chida et al., 2014, Mol Gen. & Gen. Med., 2:229-239.)

Naturally-occurring antibodies are multimeric proteins that contain four polypeptide chains (FIG. 1). Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (V_{L} in FIG. 1) and one constant region (C_{L} in FIG. 1). The heavy chain consists of one variable region (V_{H} in FIG. 1) and at least three constant regions (CH₁, CH₂ and CH₃ in FIG. 1). The variable regions determine the specificity of the antibody.

Each variable region contains three hypervariable regions known as complementarity determining regions (CDRs) flanked by four relatively conserved regions known as framework regions (FRs). The three CDRs, referred to as CDR₁, CDR₂, and CDR₃, contribute to the antibody binding specificity. Naturally occurring antibodies have been used as starting material for engineered antibodies, such as chimeric antibodies and humanized antibodies.

There is a need for improved antibodies that neutralize the biological activity of human Notch3 and that can be used as therapeutic agents to treat human patients.

### SUMMARY OF THE INVENTION

The invention is based, in part, upon the discovery of antibodies that specifically bind human Notch3. Antibodies disclosed herein contain human Notch3 binding sites based on the CDRs of the anti-Notch3 antibodies described herein. The antibodies can be used as therapeutic agents, and, depending upon the intended uses, can be optimized, e.g., affinity-matured, to improve biochemical properties (e.g., affinity and/or specificity), to improve biophysical properties (e.g., aggregation, stability, precipitation, and/or non-specific interactions), and/or to reduce or eliminate immunogenicity, when administered to a human subject.

The disclosed antibodies prevent or inhibit activation of human Notch3, which they do by inhibiting Notch3 from binding to Notch ligands, i.e., Jag1, Jag2, DLL1, and DLL4. The disclosed antibodies can be used to inhibit the proliferation of tumor cells in vitro and/or in vivo. When administered to a human subject with cancer, the antibodies inhibit or reduce tumor growth in the subject. When administered to a human with pulmonary arterial hypertension (PAH), the antibodies treat, reduce, or alleviate the symptoms and/or causes of PAH.

These and other aspects and advantages of the invention are illustrated by the following figures, detailed description and claims.

### DESCRIPTION OF THE DRAWINGS

The invention can be more completely understood with reference to the following drawings.

FIG. 1 is a schematic representation of a typical, naturally-occurring antibody.

FIGS. 2A-C provides the amino acid sequence corresponding to the extracellular domain (ECD; amino acids 40 to 1643) of human Notch3 (SEQ ID NO:19).

FIG. 3 provides the amino acid sequence corresponding to EGF-like repeats 1-11 (amino acids 40 to 467 of the extracellular domain shown in FIG. 2) of human Notch3 (SEQ ID NO:20).

FIG. 4 shows the amino acid sequence for the complete heavy chain variable region (SEQ ID NO:6) of antibody 28042. Sequences corresponding to CDR₁ (SEQ ID NO:3), CDR₂ (SEQ ID NO:4), and CDR₃ (SEQ ID NO:5) are identified in boxes. The unboxed sequences represent framework (FR) sequences.

FIG. 5 shows the amino acid sequence for the complete light (kappa) chain variable region (SEQ ID NO:10) of antibody 28042. Sequences corresponding to CDR₁ (SEQ ID NO:7), CDR₂ (SEQ ID NO:8), and CDR₃ (SEQ ID NO:9) are identified in boxes. The unboxed sequences represent framework (FR) sequences.

FIG. 6 shows the binding of the 28042 antibody to Fc fusion proteins of the extracellular domains of Notch1, Notch2, and Notch3, as observed by surface plasmon resonance (SPR). Binding was measured at three different dilutions. The results demonstrate that the binding affinity of the 28042 antibody for Notch3 is high while there is no appreciable binding of the 28042 antibody to Notch1 or Notch2.

FIG. 7 shows the binding of the 28042 antibody to cell surface human Notch1, human Notch2, human Notch3, and murine Notch3 as observed by FACS. The results demonstrate that the 28042 antibody binds human and murine Notch3 while there is no appreciable binding of the 28042 antibody to Notch1 or Notch2.

FIG. 8 is a Western blot that shows the level of inhibition of Jag1-induced Notch3 activation by the 28042 antibody as compared to other antibodies, as assessed by extent of intracellular domain cleavage of mNotch3 expressed in FlpIn™ cells.

FIG. 9 is a Western blot that shows the level of inhibition of DLL4-induced human Notch3 receptor activation, as assessed by Notch3 intracellular domain cleavage in NCI-H838 cells plated on ligand in the presence of the 28042 antibody and other antibodies.

FIGS. 10A-B show the inhibition of ligand-induced Notch3 receptor activation, as assayed by Notch3 intracellular domain cleavage. FIG. 10A is a Western blot that shows the level of inhibition of Jag1, Jag2, and DLL4 induced Notch3 activation by the 28042 antibody as assessed by extent of intracellular domain cleavage of murine Notch3 expressed in FlpIn™ cells. FIG. 10B is a Western blot that shows the level of inhibition of Jag1, Jag2, and DLL4 induced Notch3 activation by the 28042 antibody as assessed by extent of intracellular domain cleavage of human Notch3 expressed in FlpIn™ cells.

FIG. 11 is a dose-response curve based on a human Notch3 reporter assay showing that the 28042 antibody inhibits functional Notch3 activation, as measured by Notch3-dependent reporter gene expression in the presence of the Fc-bound ligand Jag2 (Jag2-Fc). The graph shows the inhibition of Jag2-Fc-stimulated reporter activity (% Inhibition) by antibody the 28042 antibody in stably transduced cells as a function of the amount of the 28042 antibody.

FIGS. 12A-B show the inhibition of tumor growth by the 28042 antibody in a Notch3-mediated mouse tumor model. FIG. 12A shows the tumor volume over time (in days) for mice treated with hIgG or the 28042 antibody. The 28042 antibody showed significant tumor growth inhibition compared to the IgG control. FIG. 12B depicts results of Western blotting performed on lysate of harvested tumors using an antibody specific for the Notch3 C-terminus in order to detect the extent of Notch intracellular domain cleavage. β-tubulin was used as a loading control.

FIG. 13 shows an alignment of the light chain variable region amino acid sequences for the 28042 antibody and variants 28042.1-28042.10. CDRs are identified by bold lettering and boxes.

FIG. 14 shows the light chain CDRs for the 28042 antibody and variants 28042.1-28042.10.

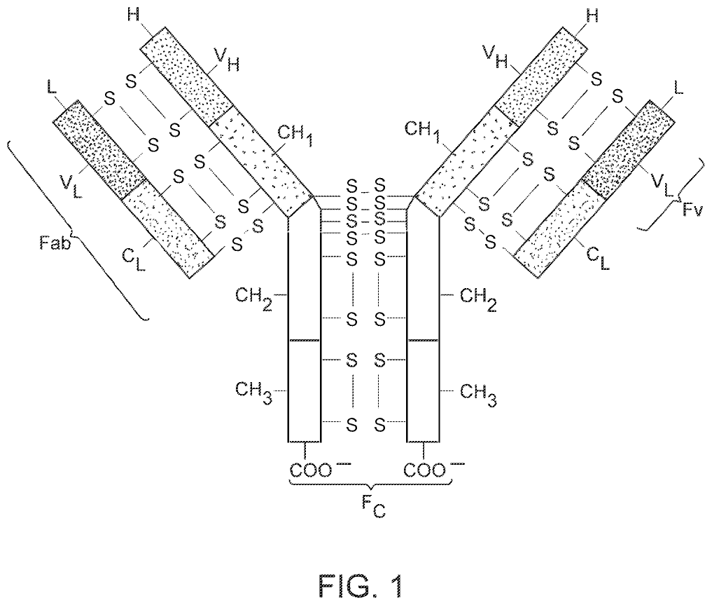

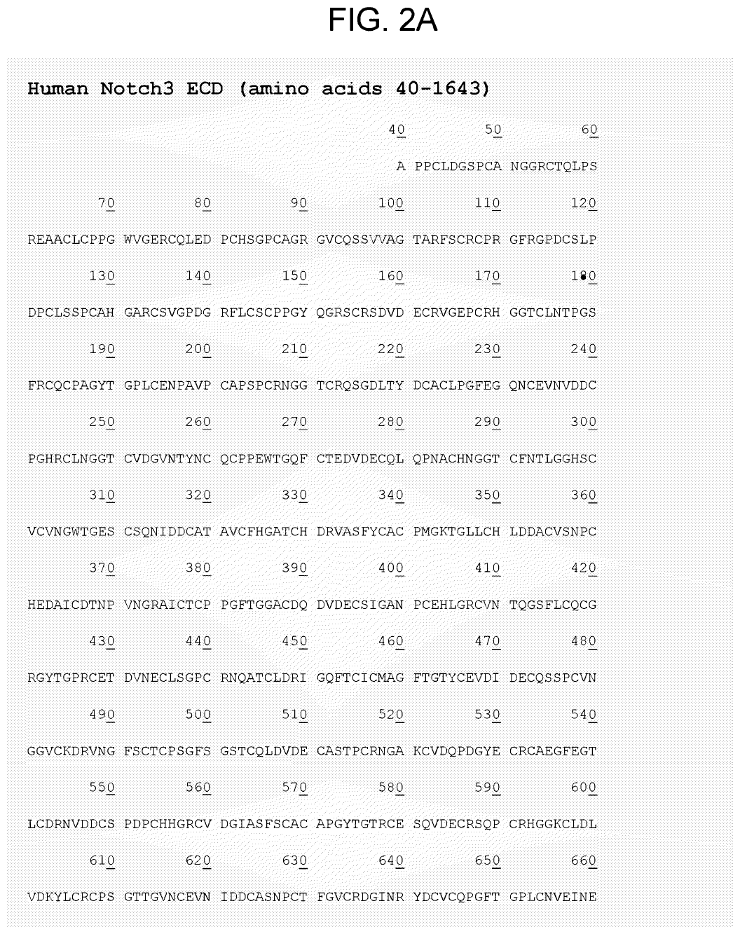

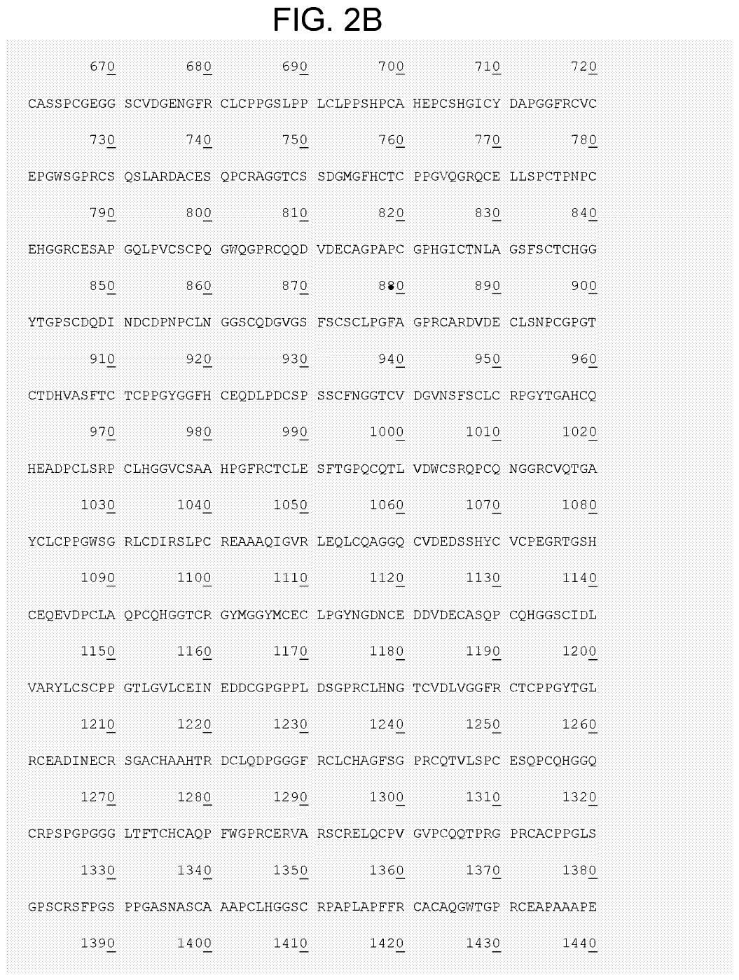

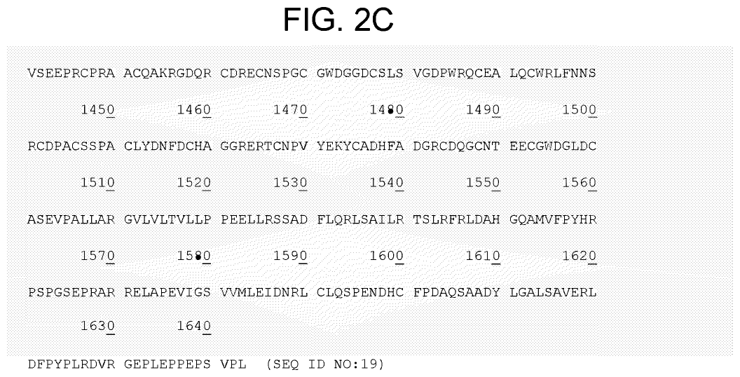

### DETAILED DESCRIPTION

The antibodies disclosed herein are based on the antigen binding sites of certain monoclonal antibodies that have been selected on the basis of binding and neutralizing the activity of human Notch3. The antibodies contain immunoglobulin variable region CDR sequences that define a binding site for human Notch3.

Because of the neutralizing activity of these antibodies, they can be used to inhibit the growth and/or proliferation of certain cancer cells and tumors and for treating pulmonary arterial hypertension (PAH) or other conditions where the Notch3 pathway is involved. When used as a therapeutic agent, the antibodies can be optimized, e.g., affinity-matured, to improve biochemical properties and/or biophysical properties, and/or to reduce or eliminate immunogenicity when administered to a human patient. Various features and aspects of the invention are discussed in more detail below.

As used herein, unless otherwise indicated, the term “antibody” means an intact antibody (e.g., an intact monoclonal antibody) or antigen-binding fragment of an antibody, including an intact antibody or antigen-binding fragment that has been modified, engineered or chemically conjugated. Examples of antibodies that have been modified or engineered are chimeric antibodies, humanized antibodies, and multispecific antibodies (e.g., bispecific antibodies). Examples of antigen-binding fragments include Fab, Fab′, F(ab′)₂, Fv, single chain antibodies (e.g., scFv), minibodies, and diabodies. An antibody conjugated to a toxin moiety is an example of a chemically conjugated antibody.

I. Antibodies that Bind Human Notch3

As disclosed herein, the antibodies may comprise: (a) an immunoglobulin heavy chain variable region comprising the structure CDR_{H1}-CDR_{H2}-CDR_{H3} and (b) an immunoglobulin light chain variable region comprising the structure CDR_{L1}-CDR_{L2}-CDR_{L3}, wherein the heavy chain variable region and the light chain variable region together define a single binding site for binding human Notch3.

In some embodiments, the antibody comprises: (a) an immunoglobulin heavy chain variable region comprising the structure CDR_{H1}-CDR_{H2}-CDR_{H3} and (b) an immunoglobulin light chain variable region, wherein the heavy chain variable region and the light chain variable region together define a single binding site for binding hNotch3. A CDR_{H1} Comprises the amino acid sequence of SEQ ID NO: 3; a CDR_{H2} comprises the amino acid sequence of SEQ ID NO: 4 and a CDR_{H3} comprises the amino acid sequence of SEQ ID NO: 5. The CDR_{H1}, CDR_{H2}, and CDR_{H3} sequences are interposed between immunoglobulin FR sequences.

In some embodiments, the antibody comprises (a) an immunoglobulin light chain variable region comprising the structure CDR_{L1}-CDR_{L2}-CDR_{L3}, and (b) an immunoglobulin heavy chain variable region, wherein the IgG light chain variable region and the IgG heavy chain variable region together define a single binding site for binding hNotch3. A CDR_{L1} comprises the amino acid sequence of SEQ ID NO: 7, a CDR_{L2} comprises the amino acid sequence of SEQ ID NO: 8; and a CDR_{L3} comprises the amino acid sequence of SEQ ID NO: 9. The CDR_{L1}, CDR_{L2}, and CDR_{L3} sequences are interposed between immunoglobulin FR sequences.

In some embodiments, the antibody comprises (a) an immunoglobulin light chain variable region comprising the structure CDR_{L1}-CDR_{L2}-CDR_{L3}, and (b) an immunoglobulin heavy chain variable region, wherein the IgG light chain variable region and the IgG heavy chain variable region together define a single binding site for binding hNotch3. A CDR_{L1} comprises the amino acid sequence of SEQ ID NO: 7 or SEQ ID NO:21, a CDR_{L2} comprises the amino acid sequence of SEQ ID NO: 8 or SEQ ID NO: 22; and a CDR_{L3} comprises the amino acid sequence of SEQ ID NO: 9. The CDR_{L1}, CDR_{L2}, and CDR_{L3} sequences are interposed between immunoglobulin FR sequences.

In some embodiments, the antibody comprises: (a) an immunoglobulin heavy chain variable region comprising the structure CDR_{H1}-CDR_{H2}-CDR_{H3} and (b) an immunoglobulin light chain variable region comprising the structure CDR_{L1}-CDR_{L2}-CDR_{L3}, wherein the heavy chain variable region and the light chain variable region together define a single binding site for binding hNotch3. The CDR_{H1} is the amino acid sequence of SEQ ID NO: 3; the CDR_{H2} is the amino acid sequence of SEQ ID NO: 4; and the CDR_{H3} is the amino acid sequence of SEQ ID NO: 5. The CDR_{L1} is the amino acid sequence of SEQ ID NO: 7; the CDR_{L2} is the amino acid sequence of SEQ ID NO: 8; and the CDR_{L3} is the amino acid sequence of SEQ ID NO: 9.

In some embodiments, the antibody comprises: (a) an immunoglobulin heavy chain variable region comprising the structure CDR_{H1}-CDR_{H2}-CDR_{H3} and (b) an immunoglobulin light chain variable region comprising the structure CDR_{L1}-CDR_{L2}-CDR_{L3}, wherein the heavy chain variable region and the light chain variable region together define a single binding site for binding hNotch3. The CDR_{H1} is the amino acid sequence of SEQ ID NO: 3; the CDR_{H2} is the amino acid sequence of SEQ ID NO: 4; and the CDR_{H3} is the amino acid sequence of SEQ ID NO: 5. The CDR_{L1} is the amino acid sequence of SEQ ID NO: 7 or SEQ ID NO: 21; the CDR_{L2} is the amino acid sequence of SEQ ID NO: 8 or SEQ ID NO: 22; and the CDR_{L3} is the amino acid sequence of SEQ ID NO: 9.

In other embodiments, the antibodies disclosed herein comprise an immunoglobulin heavy chain variable region and an immunoglobulin light chain variable region. In some embodiments, the antibody comprises an immunoglobulin heavy chain variable region comprising the amino acid sequence of SEQ ID NO: 6, and an immunoglobulin light chain variable region.

In other embodiments, the antibody comprises an immunoglobulin light chain variable region comprising the amino acid sequence of SEQ ID NO: 10 and an immunoglobulin heavy chain variable region.

In other embodiments, the antibody comprises an immunoglobulin light chain variable region comprising the amino acid sequence selected from the group consisting of SEQ ID NO: 10, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 37, SEQ ID NO: 38, and SEQ ID NO: 39 and an immunoglobulin heavy chain variable region.

In some embodiments, the antibody comprises an immunoglobulin heavy chain variable region comprising the amino acid sequence of SEQ ID NO: 6, and an immunoglobulin light chain variable region comprising the amino acid sequence selected from the group consisting of SEQ ID NO: 10, SEQ ID NO: 23, SEQ ID NO: 24, SEQ ID NO: 25, SEQ ID NO: 26, SEQ ID NO: 27, SEQ ID NO: 28, SEQ ID NO:29, SEQ ID NO:37, SEQ ID NO:38, and SEQ ID NO:39.

In some embodiments, the antibody comprises an immunoglobulin heavy chain variable region comprising the amino acid sequence of SEQ ID NO: 6, and an immunoglobulin light chain variable region comprising the amino acid sequence of SEQ ID NO: 10.

In some embodiments, the antibody comprises an immunoglobulin heavy chain variable region comprising the amino acid sequence of SEQ ID NO: 6, and an immunoglobulin light chain variable region comprising the amino acid sequence of SEQ ID NO: 10, wherein the amino acid sequence of SEQ ID NO: 10 includes a mutation at one or more of positions 31, 50, 62, 65, 106, and 107. In some embodiments, the amino acid sequence of SEQ ID NO:10 includes one or more mutations selected from the group consisting of N31S, G50A, V62F, T65S F106I, and E107K.

In certain embodiments, the antibodies disclosed herein comprise an immunoglobulin heavy chain and an immunoglobulin light chain. In some embodiments, the antibody comprises an immunoglobulin heavy chain comprising the amino acid sequence of SEQ ID NO: 1, and an immunoglobulin light chain.

In other embodiments, the antibody comprises an immunoglobulin light chain comprising the amino acid sequence of SEQ ID NO: 2, and an immunoglobulin heavy chain.

In other embodiments, the antibody comprises an immunoglobulin light chain comprising an amino acid sequence selected from the group consisting of SEQ ID NO: 2, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 40, SEQ ID NO: 41, and SEQ ID NO: 42 and an immunoglobulin heavy chain.

In some embodiments, the antibody comprises an immunoglobulin heavy chain comprising the amino acid sequence of SEQ ID NO: 1, and an immunoglobulin light chain comprising the amino acid sequence of SEQ ID NO: 2.

In some embodiments, the antibody comprises an immunoglobulin heavy chain comprising the amino acid sequence of SEQ ID NO: 1, and an immunoglobulin light chain comprising an amino acid sequence selected from the group consisting of SEQ ID NO: 2, SEQ ID NO: 30, SEQ ID NO: 31, SEQ ID NO:32, SEQ ID NO: 33, SEQ ID NO: 34, SEQ ID NO: 35, SEQ ID NO: 36, SEQ ID NO: 40, SEQ ID NO: 41, and SEQ ID NO: 42.

In some embodiments, the antibody comprises an immunoglobulin heavy chain comprising the amino acid sequence of SEQ ID NO: 1, and an immunoglobulin light chain comprising the amino acid sequence of SEQ ID NO: 2, wherein the amino acid sequence of SEQ ID NO: 2 includes a mutation at one or more of positions 31, 50, 62, 65, 106, and 107. In some embodiments, the amino acid sequence of SEQ ID NO:10 includes one or more mutations selected from the group consisting of N31S, G50A, V62F, T65S, F106I, and E107K.

In certain embodiments, an isolated antibody that binds hNotch3 comprises an immunoglobulin heavy chain variable region comprising an amino acid sequence that is at least 70%, 75%, 80%, 85%, 90%, 95%, 96%, 97%, 98%, or 99% identical to the entire variable region or the framework region sequence of SEQ ID NO: 6. In certain embodiments, an isolated antibody that binds hNotch3 comprises an immunoglobulin light chain variable region comprising an amino acid sequence that is at least 70%, 75%, 80%, 85%, 90%, 95%, 96%, 97%, 98%, or 99% identical to the entire variable region or the framework region sequence of SEQ ID NO: 10. In certain embodiments, an isolated antibody that binds hNotch3 comprises an immunoglobulin light chain variable region comprising an amino acid sequence that is at least 70%, 75%, 80%, 85%, 90%, 95%, 96%, 97%, 98%, or 99% identical to the entire variable region or the framework region sequence of SEQ ID NO: 23, 24, 25, 26, 27, 28, 29, 37, 38, or 39.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; —W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In each of the foregoing embodiments, it is contemplated herein that immunoglobulin heavy chain variable region sequences and/or light chain variable region sequences that together bind hNotch3 may contain amino acid alterations (e.g., at least 1, 2, 3, 4, 5, or 10 amino acid substitutions, deletions, or additions) in the framework regions of the heavy and/or light chain variable regions.

In certain embodiments, the antibody binds hNotch3 with a K_{D} of 20 nM, 15 nM, 10 nM, 9 nM, 8 nM, 7 nM, 6 nM, 5 nM, 4 nM, 3 nM, 2 nM, 1 nM or lower. Unless otherwise specified, K_{D} values are determined by surface plasmon resonance or bio-layer interferometry methods under the conditions described in Example 2.

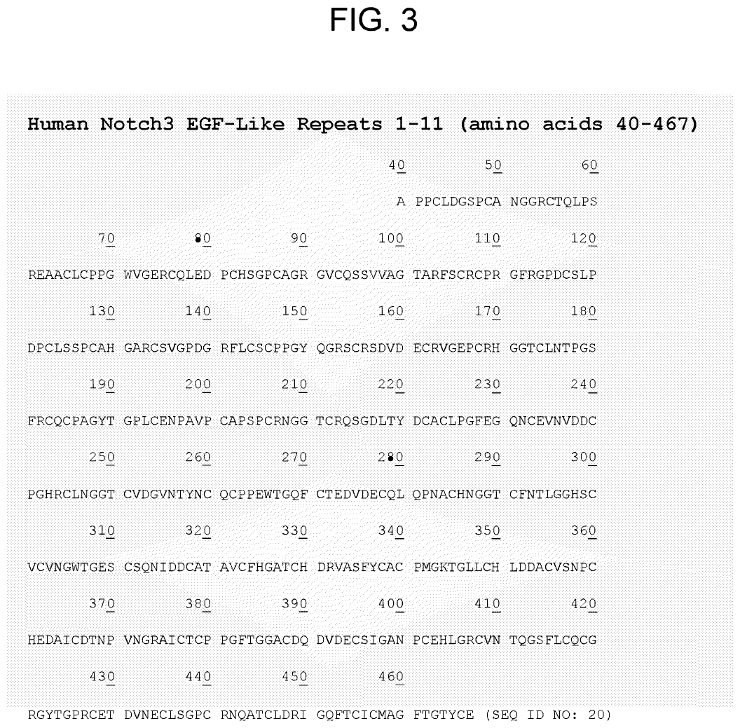

In some embodiments, monoclonal antibodies bind to the same epitope on hNotch3 as that bound by the 28042 antibody. In some embodiments, monoclonal antibodies compete for binding to hNotch3 with the 28042 antibody. For example, monoclonal antibodies may compete for binding to the extracellular domain (ECD) of Notch3 with the 28042 antibody. In another example, monoclonal antibodies may compete for binding to EGF-like repeats 1-11 of human Notch3 with the 28042 antibody (amino acid sequence corresponding to EGF-like repeats 1-11 of human Notch3 is shown in FIG. 3).

Competition assays for determining whether an antibody binds to the same epitope as, or competes for binding with, the 28042 antibody are known in the art. Exemplary competition assays include immunoassays (e.g., ELISA assays, RIA assays), BIAcore analysis, biolayer interferometry, and flow cytometry.

Typically, a competition assay involves the use of an antigen (e.g., a human Notch3 protein or fragment thereof) bound to a solid surface or expressed on a cell surface, a test Notch3-binding antibody and a reference antibody (e.g., the 28042 antibody). The reference antibody is labeled and the test antibody is unlabeled. Competitive inhibition is measured by determining the amount of labeled reference antibody bound to the solid surface or cells in the presence of the test antibody. Usually the test antibody is present in excess (e.g., 1×, 5×, 10×, 20× or 100×). Antibodies identified by competition assay (i.e., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

In an exemplary competition assay, a reference Notch3 antibody (e.g., the 28042 antibody) is biotinylated using commercially available reagents. The biotinylated reference antibody is mixed with serial dilutions of the test antibody or unlabeled reference antibody (self-competition control) resulting in a mixture of various molar ratios (e.g., 1×, 5×, 10×, 20× or 100×) of test antibody (or unlabeled reference antibody) to labeled reference antibody. The antibody mixture is added to a human Notch3 (e.g., extracellular domain of human Notch3) polypeptide coated-ELISA plate. The plate then is washed and HRP (horseradish peroxidase)-strepavidin is added to the plate as the detection reagent. The amount of labeled reference antibody bound to the target antigen is detected following addition of a chromogenic substrate (e.g., TMB (3,3′,5,5′-tetramethylbenzidine) or ABTS (2,2″-di-(3-ethylbenzthiazoline-6-sulfonate)), which are well-known in the art. Optical density readings (OD units) are measured using a SpectraMax M2 spectrometer (Molecular Devices). OD units corresponding to zero percent inhibition are determined from wells without any competing antibody. OD units corresponding to 100% inhibition, i.e., the assay background are determined from wells without any labeled reference antibody or test antibody. Percent inhibition of labeled reference antibody to Notch3 by the test antibody (or the unlabeled reference antibody) at each concentration is calculated as follows: % inhibition=(1−(OD units−100% inhibition)/(0% inhibition−100% inhibition))*100. Persons skilled in the art will appreciate that the competition assay can be performed using various detection systems well-known in the art.

A competition assay can be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

### II. Antibody Production

Methods for producing antibodies of the invention are known in the art. For example, DNA molecules encoding light chain variable regions and/or heavy chain variable regions can be chemically synthesized using the sequence information provided herein. Synthetic DNA molecules can be ligated to other appropriate nucleotide sequences, including, e.g., constant region coding sequences, and expression control sequences, to produce conventional gene expression constructs encoding the desired antibody. Production of defined gene constructs is within routine skill in the art.

Nucleic acids encoding desired antibodies can be incorporated (ligated) into expression vectors, which can be introduced into host cells through conventional transfection or transformation techniques. Exemplary host cells are E. coli cells, Chinese hamster ovary (CHO) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon, and, optionally, may contain enhancers, and various introns. This expression vector optionally contains sequences encoding all or part of a constant region, enabling an entire, or a part of, a heavy or light chain to be expressed. The gene construct can be introduced into eukaryotic host cells using conventional techniques. The host cells express V_{L} or V_{H} fragments, V_{L}V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In other embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In still other embodiments, a host cell is co-transfected with more than one expression vector (e.g., one expression vector encoding a polypeptide comprising an entire, or part of, a heavy chain or heavy chain variable region, and another expression vector encoding a polypeptide comprising an entire, or part of, a light chain or light chain variable region).

A polypeptide comprising an immunoglobulin heavy chain variable region or light chain variable region can be produced by growing (culturing) a host cell transfected with an expression vector encoding such variable region, under conditions that permit expression of the polypeptide. Following expression, the polypeptide can be harvested and purified or isolated using techniques known in the art, e.g., affinity tags such as glutathione-S-transferase (GST) and histidine tags.

A monoclonal antibody that binds human Notch3, or an antigen-binding fragment of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The intact antibody (or antigen-binding fragment of the antibody) can be harvested and purified or isolated using techniques known in the art, e.g., Protein A, Protein G, affinity tags such as glutathione-S-transferase (GST) and histidine tags. It is within ordinary skill in the art to express the heavy chain and the light chain from a single expression vector or from two separate expression vectors.

### III. Antibody Modifications

Human monoclonal antibodies can be isolated or selected from phage display libraries including immune, naïve and synthetic libraries. Antibody phage display libraries are known in the art, see, e.g., Hoet et al., NATURE BIOTECH. 23:344-348, 2005; Soderlind et al., NATURE BIOTECH. 18:852-856, 2000; Rothe et al., J. MOL. BIOL. 376:1182-1200, 2008; Knappik et al., J. MOL. BIOL. 296:57-86, 2000; and Krebs et al., J. IMMUNOL. METH. 254:67-84, 2001. When used as a therapeutic, human antibodies isolated by phage display may be optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In some embodiments, isolated human antibodies contain one or more somatic mutations. In these cases, antibodies can be modified to a human germline sequence to optimize the antibody (i.e., a process referred to as germlining).

Generally, an optimized antibody has at least the same, or substantially the same, affinity for the antigen as the non-optimized (or parental) antibody from which it was derived. Preferably, an optimized antibody has a higher affinity for the antigen when compared to the parental antibody.

Human antibody fragments (e.g., parental and optimized variants) can be engineered to contain certain constant (i.e., Fc) regions with a specified effector function (e.g., antibody-dependent cellular cytotoxicity (ADCC)). Human constant regions are known in the art.

If the antibody is for use as a therapeutic, it can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

The antibody can be conjugated to an effector moiety such as a small molecule toxin or a radionuclide using standard in vitro conjugation chemistries. If the effector moiety is a polypeptide, the antibody can be chemically conjugated to the effector or joined to the effector as a fusion protein. Construction of fusion proteins is within ordinary skill in the art.

### IV. Use of Antibodies

Antibodies disclosed herein can be used to treat various forms of cancer, e.g., leukemia, breast cancer, cervical cancer, colorectal cancer, lung cancer, pancreatic cancer, prostate cancer, gastric cancer, head and neck cancer, endometrial cancer and ovarian cancer. The cancer cells are exposed to a therapeutically effective amount of the antibody so as to inhibit or reduce proliferation of the cancer cells. In some embodiments, the antibodies inhibit cancer cell proliferation by at least 40%, 50%, 60%, 70%, 80%, 90%, 95%, 98%, 99% or 100% relative to a control antibody that does not bind human Notch3 with the same avidity as the 28042 antibody.

In some embodiments, the 28042 antibody or a variant thereof is used in therapy. For example, the 28042 antibody can be used to inhibit tumor growth in a mammal (e.g., a human patient). In some embodiments, use of the antibody to inhibit tumor growth in a mammal comprises administering to the mammal a therapeutically effective amount of the antibody. In other embodiments, the 28042 antibody or a variant thereof can be used for inhibiting proliferation of a tumor cell. In other embodiments, the 28042 antibody or a variant thereof is used to treat or prevent pulmonary arterial hypertension (PAH).

In some embodiments, the disclosed antibodies may inhibit or reduce proliferation of a tumor cell by inhibiting binding of human Notch3 to a ligand, e.g., Jag1, Jag2, DLL1, and DLL4. The disclosed antibodies can be used in a method to inhibit tumor growth in a human patient. The method comprises administering to the patient a therapeutically effective amount of the antibody.

Cancers associated with Notch3 overexpression and/or activation include, but are not limited to, breast cancer, ovarian cancer, prostate cancer, cervical cancer, lung cancer, brain cancer (e.g., glioblastoma, astrocytoma, neuroblastoma), melanomas, gastrointestinal cancers (e.g., colorectal, pancreatic, and gastric), head and neck cancer, sarcomas (e.g. rhabdomyosarcoma, osteosarcoma), and hematopoietic cell cancers, (e.g., multiple myeloma, leukemia, e.g., precursor T acute lymphoblastic leukemia (T-ALL), precursor B acute lymphoblastic leukemia (B-ALL) and B-cell chronic lymphoblastic leukemia (B-CLL)).

As used herein, “treat”, “treating” and “treatment” mean the treatment of a disease in a mammal, e.g., in a human. This includes: (a) inhibiting the disease, i.e., arresting its development; and (b) relieving the disease, i.e., causing regression of the disease state.

Generally, a therapeutically effective amount of active component is in the range of 0.1 mg/kg to 100 mg/kg, e.g., 1 mg/kg to 100 mg/kg, 1 mg/kg to 10 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody, and the disease being treated. Exemplary dosing frequencies are once per day, once per week and once every two weeks. A preferred route of administration is parenteral, e.g., intravenous infusion. Formulation of monoclonal antibody-based drugs is within ordinary skill in the art. In some embodiments, a monoclonal antibody is lyophilized, and then reconstituted in buffered saline, at the time of administration.

For therapeutic use, an antibody preferably is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing antibodies disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. A preferred route of administration for monoclonal antibodies is IV infusion. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

### EXAMPLES

The following Examples are merely illustrative and are not intended to limit the scope or content of the invention in any way.

### Example 1: Anti-Notch3 Antibodies

This Example describes the production of anti-hNotch3 monoclonal antibodies.

### A. Isolation of Anti-Notch3-Antibodies by Phage Display

Anti-hNotch3 antibodies are isolated from a phage display library constructed from human heavy chain and light chain variable region genes. More specifically, the anti-hNotch3 antibodies are isolated using a phagemid antibody library constructed from human B-cell donor light chain variable and constant genes paired with a single human heavy chain framework consisting of human B-cell donor heavy chain variable region CDR₃ genes and a diversity of synthetic heavy chain variable region CDR₁ and CDR₂ sequences (Dyax Corporation, Burlington, MA).

An anti-hNotch3 antibody (the 28042 antibody) was isolated from a human antibody phage display library following three phage display selection rounds using diminishing amounts of a biotinylated recombinant concatemer of alternating human and murine Notch 3 EGF-like Repeats 1-11 (FIG. 3) as the antigen. 200 pmol of the antigen was used in round one, followed by 50 and 5 pmol in rounds two and three, respectively. At the start of each round of selection, the phage library was depleted with biotinylated human IgG1 Fc. Each round of selection was followed by infection of TG1 E. coli (Agilent Technologies, Santa Clara, CA), rescue with M13K07 helper phage (Life Technologies, Grand Island, NY), and phage amplification to enrich the input for each subsequent round. Colonies resulting from the third round of selection were screened by ELISA for binding to both recombinant human and mouse Notch3. All positive clones were sequenced. Clones with unique sequences were expressed as Fabs, Protein A purified, and screened for function. Functional antibodies were converted to full length IgG1 antibodies and further tested for function.

Additionally, non-human light chain residues of the 28042 antibody were individually changed to human germline sequences to create additional light chain variants. For example, the 28042.1 antibody contains the light chain CDR mutation N31S; the 28042.2 antibody contains the light chain CDR mutation G50A; the 28042.3 antibody contains the light chain framework mutation F106I; the 28042.4 antibody contains the light chain framework mutation E107K; the 28042.5 antibody contains the light chain CDR and framework mutations N31S and G50A; the 28042.6 antibody contains the light chain framework mutations F106I and E107K; the 28042.7 antibody contains the light chain CDR and framework mutations N31S, G50A, F106I, E107K; the 28042.8 antibody contains the light chain framework mutation V62F; the 28042.9 antibody contains the light chain framework mutation T65S; and the 28042.10 antibody contains the light chain CDR and framework mutations N31S, G50A, V62F, and T65S. An antibody according to the invention may contain 1 or more of the aforementioned light chain framework and/or CDR mutations.

### B. Sequences of Anti-Notch3 Antibodies

The human heavy chains are subcloned into pEE6.4 (Lonza, Basel, Switzerland) via HindIII and EcoRI sites using In-Fusion™ PCR cloning (Clontech, Mountain View, CA). The human kappa light chains are subcloned into pEE14.4 (Lonza) via HindIII and EcoRI sites using In-Fusion™ PCR cloning.

Antibody is either expressed by transient transfection of 293T cells with separate heavy and light chain expressing vectors or by stable transfection of CHOK1SV cells (Lonza) with a single vector expressing heavy and light chains. Antibodies are purified for subsequent analysis. A single expression vector can be constructed by combining pEE6.4 and pEE14.4 based vectors. First, pEE6.4 containing full length human heavy chain cDNA is digested with NotI and SalI to create a fragment containing the hCMV-MIE promoter+full length human heavy chain cDNA+SV40 poly A. This fragment is inserted into the pEE14.4 vector already containing full length human light chain cDNA via NotI/SalI sites, thus creating an expression vector that simultaneously expresses heavy and light chains. The combined heavy and light chain vector is linearized and transfected into CHOK1SV cells. Stable clones are selected in the presence of methionine sulfoximine. Binding of the human antibodies to human Notch3 can be measured as described below.

The nucleic acid sequences encoding and the protein sequences defining variable regions of the human 28042 antibodies are summarized below (amino terminal signal peptide sequences are not shown). In the amino acid sequences, CDR sequences (using the Kabat system) are shown in bold and are underlined.

Nucleic Acid Sequence Encoding the Heavy Chain of the 28042 Antibody
(SEQ ID NO: 11)
gaagttcaattgttagagtctggtggcggtcttgttcagcctggtggttctttacgtctttctt
gcgctgcttccggattcactttctctgattacatgatgtcttgggttcgccaagctcctggtaa
aggtttggagtgggtttcttggatccgttcttctggtggcactactctttatgctgactccgtt
aaaggtcgcttcactatctctagagacaactctaagaatactctctacttgcagatgaacagct
taagggctgaggacacagccacatattactgtgcgagagtcggtggtgggaccacgggttatgc
ttttgatatctggggccaagggacaatggtcaccgtctcaagcgcctcaacaaaaggaccaagt
gtgttcccactcgcccctagcagcaagagtacatccgggggcactgcagcactcggctgcctcg
tcaaggattattttccagagccagtaaccgtgagctggaacagtggagcactcacttctggtgt
ccatacttttcctgctgtcctgcaaagctctggcctgtactcactcagctccgtcgtgaccgtg
ccatcttcatctctgggcactcagacctacatctgtaatgtaaaccacaagcctagcaatacta
aggtcgataagcgggtggaacccaagagctgcgacaagactcacacttgtcccccatgccctgc
ccctgaacttctgggcggtcccagcgtctttttgttcccaccaaagcctaaagatactctgatg
ataagtagaacacccgaggtgacatgtgttgttgtagacgtttcccacgaggacccagaggtta
agttcaactggtacgttgatggagtcgaagtacataatgctaagaccaagcctagagaggagca
gtataatagtacataccgtgtagtcagtgttctcacagtgctgcaccaagactggctcaacggc
aaagaatacaaatgcaaagtgtccaacaaagcactcccagcccctatcgagaagactattagta
aggcaaaggggcagcctcgtgaaccacaggtgtacactctgccacccagtagagaggaaatgac
aaagaaccaagtctcattgacctgcctggtgaaaggcttctaccccagcgacatcgccgttgag
tgggagagtaacggtcagcctgagaacaattacaagacaacccccccagtgctggatagtgacg
ggtctttctttctgtacagtaagctgactgtggacaagtcccgctggcagcagggtaacgtctt
cagctgttccgtgatgcacgaggcattgcacaaccactacacccagaagtcactgagcctgagc
ccagggaag (1353 nucleotides)
Protein Sequence Defining the Heavy Chain of the 28042 Antibody
(SEQ ID NO: 1)
Evqllesggglvqpggslrlscaasgftfsdymmswvrqapgkglewvswirssggttlyadsv
kgrftisrdnskntlylqmnslraedtatyycarvgggttgyafdiwgqgtmvtvssastkgps
vfplapsskstsggtaalgclvkdyfpepvtvswnsgaltsgvhtfpavlqssglyslssvvtv
pssslgtqtyicnvnhkpsntkvdkrvepkscdkthtcppcpapellggpsvflfppkpkdtlm
isrtpevtcvvvdvshedpevkfnwyvdgvevhnaktkpreeqynstyrvvsvltvlhqdwlng
keykckvsnkalpapiektiskakgqprepqvytlppsreemtknqvsltclvkgfypsdiave
wesngqpennykttppvldsdgsfflyskltvdksrwqqgnvfscsvmhealhnhytqkslsls
pgk (451 amino acids)
Nucleic Acid Sequence Encoding the Heavy Chain Variable Region of
the 28042 Antibody
(SEQ ID NO: 17)
gaagttcaat tgttagagtc tggtggcggt cttgttcagc ctggtggttc 50
tttacgtctt tcttgcgctg cttccggatt cactttctct gattacatga 100
tgtcttgggt tcgccaagct cctggtaaag gtttggagtg ggtttcttgg 150
atccgttctt ctggtggcac tactctttat gctgactccg ttaaaggtcg 200
cttcactatc tctagagaca actctaagaa tactctctac ttgcagatga 250
acagcttaag ggctgaggac acagccacat attactgtgc gagagtcggt 300
ggtgggacca cgggttatgc ttttgatatc tggggccaag ggacaatggt 350
caccgtctca agc 363
Protein Sequence Defining the Heavy Chain Variable Region of the
28042 Antibody
(SEQ ID NO: 6)
evqllesggg lvqpggslrl scaasgftfs dymmswvrqa pgkglewvsw 50
irssggttly adsvkgrfti srdnskntly lqmnslraed tatyycarvg 100
ggttgyafdiwgqgtmvtvs s 121
Nucleic Acid Sequence Encoding the Kappa Chain of the 28042 Antibody
(SEQ ID NO: 12)
gacatccaga tgacccagtc tccatcctcc ctgtctgcat ctgtaggaga 50
cagagtcacc atcacttgcc gggcaagtca gagcattagc aactatttaa 100
attggtatca gcagaaacca gggaaagccc ctaagctcct gatctatggt 150
gcatccagtc tgcaaagtgg ggtcccatca agggtcagtg gcactggatc 200
tgggacagat ttcactctta ccatcagcag tctgcaacct gaagattttg 250
caacttacta ctgtcaacag agttacagtc cctcattcac tttcggccct 300
gggaccaaag tggatttcga acgaactgtg gctgcaccat ctgtgttcat 350
ctttccacca agtgatgagc aactgaagtc tggtactgct tcagtcgtgt 400
gtctgctgaa caatttctac cctcgagaag ccaaagtcca atggaaggta 450
gacaacgcac tgcagtccgg caatagccaa gaatcagtta ccgaacagga 500
ttcaaaggac agtacatatt ccctgagcag cactctgacc ctgtcaaagg 550
ccgattacga gaaacacaag gtctatgctt gcgaagtgac acatcaggga 600
ctgtccagcc cagtgacaaa atcttttaac cgtggggagt gt 642
Protein Sequence Defining the Kappa Chain of the 28042 Antibody
(SEQ ID NO: 2)
digmtgspss lsasvgdrvt itcrasqsis nylnwyqqkp gkapklliyg 50
asslqsgvps rvsgtgsgtd ftltisslqp edfatyycqq syspsftfgp 100
gtkvdfertv aapsvfifpp sdeqlksgta svvcllnnfy preakvqwkv 150
dnalqsgnsq esvtegdskd styslsstlt lskadyekhk vyacevthqg 200
lsspvtksfn rgec 214
Nucleic Acid Sequence Encoding the Kappa Chain Variable Region of
the 28042 Antibody
(SEQ ID NO: 18)
gacatccaga tgacctagtc tccatcctcc ctgtctgcat ctgtaggaga 50
cagagtcacc atcacttgcc gggcaagtca gagcattagc aactatttaa 100
attggtatca gcagaaacca gggaaagccc ctaagctcct gatctatggt 150
gcatccagtc tgcaaagtgg ggtcccatca agggtcagtg gcactggatc 200
tgggacagat ttcactctta ccatcagcag tctgcaacct gaagattttg 250
caacttacta ctgtcaacag agttacagtc cctcattcac tttcggccct 300
gggaccaaag tggatttcga a 321
Protein Sequence Defining the Kappa Chain Variable Region of the
28042 Antibody
(SEQ ID NO: 10)
diqmtqspss lsasvgdrvt itcrasqsis nylnwyqqkp gkapklliyg 50
asslqsgvps rvsgtgsgtd ftltisslqp edfatyycqq syspsftfgp 100
gtkvdfe 107

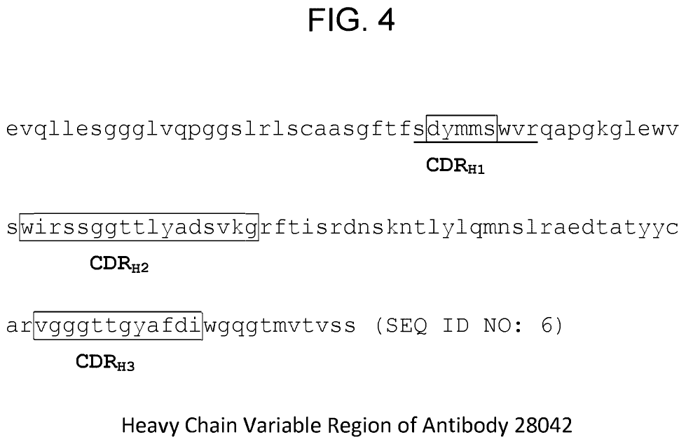

The amino acid sequence defining the immunoglobulin heavy chain variable region for the antibodies produced in Example 1 is shown in FIG. 4. Amino terminal signal peptide sequences (for proper expression/secretion) are not shown. CDR₁, CDR₂, and CDR₃ (using the Kabat system) are identified by boxes.

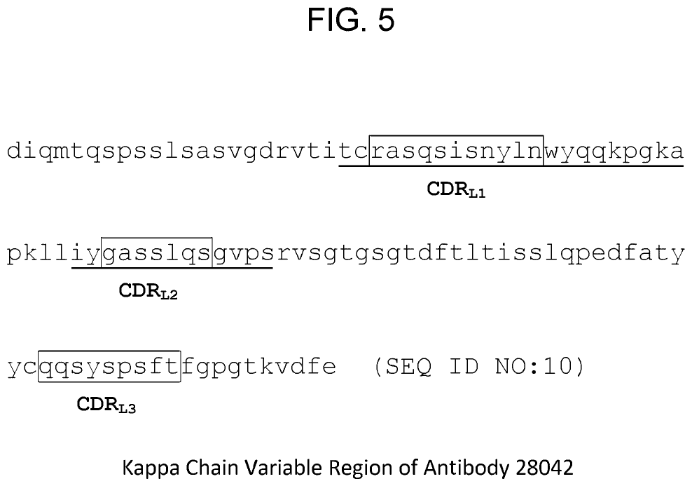

The amino acid sequences defining the immunoglobulin light chain variable regions for the antibodies in Example 1 are aligned in FIG. 5. Amino terminal signal peptide sequences (for proper expression/secretion) are not shown. CDR₁, CDR₂ and CDR₃ are identified by boxes.

Table 1 is a concordance chart showing the SEQ ID NO. of each sequence discussed in this Example.

TABLE 1

| SEQ. ID | |
|---|---|
| NO. | Nucleic Acid or Protein |
| 11 | Heavy Chain of the 28042 Antibody-nucleic acid |
| 1 | Heavy Chain of the 28042 Antibody-protein |
| 17 | Heavy Chain Variable Region of the 28042 Antibody-nucleic |
| | acid |
| 6 | Heavy Chain Variable Region of the 28042 Antibody-protein |
| 12 | Kappa (light) Chain of the 28042 Antibody-nucleic acid |
| 2 | Kappa (light) Chain of the 28042 Antibody-protein |
| 18 | Kappa (light) Chain Variable Region of the 28042 Antibody- |
| | nucleic acid |
| 10 | Kappa (light) Chain Variable Region of the 28042 Antibody- |
| | protein |

Human monoclonal antibody 28042 heavy chain CDR sequences (Kabat definition) are shown in Table 2.

TABLE 2

| | CDR1 | CDR2 | CDR3 |
|---|---|---|---|
| Kabat | DYMMS | WIRSSGGTTLYADSVKG | VGGGTTGYAFDI |
| | (SEQ ID | (SEQ ID NO: 4) | (SEQ ID NO: 5) |
| | NO: 3) | | |

Human monoclonal antibody 28042 Kappa light chain CDR sequences (Kabat definition) are shown in Table 3.

TABLE 3

| | CDR1 | CDR2 | CDR3 |
|---|---|---|---|
| Kabat | RASQSISNYLN | GASSLQS | QQSYSPSFT |
| | (SEQ ID NO: 7) | (SEQ ID NO: 8) | (SEQ ID NO: 9) |

Nucleic Acid Sequence Encoding the Human IgG1 Heavy Chain
Constant Region
(SEQ ID NO: 16)
gcctcaacaa aaggaccaag tgtgttccca ctcgccccta gcagcaagag 50
tacatccggg ggcactgcag cactcggctg cctcgtcaag gattattttc 100
cagagccagt aaccgtgagc tggaacagtg gagcactcac ttctggtgtc 150
catacttttc ctgctgtcct gcaaagctct ggcctgtact cactcagctc 200
cgtcgtgacc gtgccatctt catctctggg cactcagacc tacatctgta 250
atgtaaacca caagcctagc aatactaagg tcgataagcg ggtggaaccc 300
aagagctgcg acaagactca cacttgtccc ccatgccctg cccctgaact 350
tctgggcggt cccagcgtct ttttgttccc accaaagcct aaagatactc 400
tgatgataag tagaacaccc gaggtgacat gtgttgttgt agacgtttcc 450
cacgaggacc cagaggttaa gttcaactgg tacgttgatg gagtcgaagt 500
acataatgct aagaccaagc ctagagagga gcagtataat agtacatacc 550
gtgtagtcag tgttctcaca gtgctgcacc aagactggct caacggcaaa 600
gaatacaaat gcaaagtgtc caacaaagca ctcccagccc ctatcgagaa 650
gactattagt aaggcaaagg ggcagcctcg tgaaccacag gtgtacactc 700
tgccacccag tagagaggaa atgacaaaga accaagtctc attgacctgc 750
ctggtgaaag gcttctaccc cagcgacatc gccgttgagt gggagagtaa 800
cggtcagcct gagaacaatt acaagacaac ccccccagtg ctggatagtg 850
acgggtcttt ctttctgtac agtaagctga ctgtggacaa gtcccgctgg 900
cagcagggta acgtcttcag ctgttccgtg atgcacgagg cattgcacaa 950
ccactacacc cagaagtcac tgagcctgag cccagggaag 990
Protein Sequence Defining the Human IgG1 Heavy Chain
Constant Region
(SEQ ID NO: 13)
astkgpsvfp lapsskstsg gtaalgclvk dyfpepvtvs wnsgaltsgv 50
htfpavlqss glyslssvvt vpssslgtqt yicnvnhkps ntkvdkrvep 100
kscdkthtcp pcpapellgg psvflfppkp kdtlmisrtp evtcvvvdvs 150
hedpevkfnw yvdgvevhna ktkpreeqyn styrvvsvlt vlhqdwlngk 200
eykckvsnka lpapiektis kakgqprepq vytlppsree mtknqvsltc 250
lvkgfypsdi avewesngqp ennykttppv ldsdgsffly skltvdksrw 300
qqgnvfscsv mhealhnhyt qkslslspgk 330
Nucleic Acid Sequence Encoding the Human Kappa Light Chain
Constant Region of the 28042 Antibody
(SEQ ID NO: 15)
cgaactgtgg ctgcaccatc tgtgttcatc tttccaccaa gtgatgagca 50
actgaagtct ggtactgctt cagtcgtgtg tctgctgaac aatttctacc 100
ctcgagaagc caaagtccaa tggaaggtag acaacgcact gcagtccggc 150
aatagccaag aatcagttac cgaacaggat tcaaaggaca gtacatattc 200
cctgagcagc actctgaccc tgtcaaaggc cgattacgag aaacacaagg 250
tctatgcttg cgaagtgaca catcagggac tgtccagccc agtgacaaaa 300
tcttttaacc gtggggagtg t 321
Protein Sequence Defining the Human Kappa Light Chain
Constant Region of the 28042 Antibody_
(SEQ ID NO: 14)
rtvaapsvfi fppsdeqlks gtasvvclln nfypreakvq wkvdnalqsg 50
nsqesvteqd skdstyslss tltlskadye khkvyacevt hqglsspvtk 100
sfnrgec 107

Table 4 is a concordance chart showing the SEQ ID NO. of each sequence discussed in this Example.

TABLE 4

| SEQ | |
|---|---|
| ID | |
| NO. | Nucleic Acid or Protein |
| 11 | 28042 Heavy Human Variable Region (SEQ ID NO: 17) + Human |
| | IgG1 Constant Region (SEQ ID NO: 16)-nucleic acid |
| 1 | 28042 Heavy Human Variable Region (SEQ ID NO: 6) + Human |
| | IgG1 Constant Region (SEQ ID NO: 13)-protein |
| 12 | 28042 Kappa Human Variable Region (SEQ ID NO: 18) + Human |
| | Kappa Constant Region (SEQ ID NO: 15)-nucleic acid |
| 2 | 28042 Kappa Human Variable Region (SEQ ID NO: 10) + Human |
| | Kappa Constant Region (SEQ ID NO: 14)-protein |

The following sequences relate to variants of the 28042 antibody where certain residues in the framework region have been modified via germlining. The CDR sequences are underlined.

Protein Sequence Defining the Kappa Chain Variable Region of the
28042.1 Antibody
(SEQ ID NO: 23)
diqmtqspss lsasvgdrvt itcrasqsis Sylnwyqqkp gkapklliyg 50
asslqsgvps rvsgtgsgtd ftltisslqp edfatyycqq syspsftfgp 100
gtkvdfe 107
Protein Sequence Defining the Kappa Chain Variable Region of the
28042.2 Antibody
(SEQ ID NO: 24)
diqmtqspss lsasvgdrvt itcrasqsis nylnwyqqkp gkapklliyA 50
asslqsgvps rvsgtgsgtd ftltisslqp edfatyycqq syspsftfgp 100
gtkvdfe 107
Protein Sequence Defining the Kappa Chain Variable Region of the
28042.3 Antibody
(SEQ ID NO: 25)
diqmtqspss lsasvgdrvt itcrasqsis nylnwyqqkp gkapklliyg 50
asslqsgvps rvsgtgsgtd ftltisslqp edfatyycqq syspsftfgp 100
gtkvdIe 107
Protein Sequence Defining the Kappa Chain Variable Region of the
28042.4 Antibody
(SEQ ID NO: 26)
diqmtqspss lsasvgdrvt itcrasqsis nylnwyqqkp gkapklliyg 50
asslqsgvps rvsgtgsgtd ftltisslqp edfatyycqq syspsftfgp 100
gtkvdfK 107
Protein Sequence Defining the Kappa Chain Variable Region of the
28042.5 Antibody
(SEQ ID NO: 27)
diqmtqspss lsasvgdrvt itcrasqsis Sylnwyqqkp gkapklliyA 50
asslqsgvps rvsgtgsgtd ftltisslqp edfatyycqq syspsftfgp 100
gtkvdfe 107
Protein Sequence Defining the Kappa Chain Variable Region of the
28042.6 Antibody
(SEQ ID NO: 28)
diqmtqspss lsasvgdrvt itcrasqsis nylnwyqqkp gkapklliyg 50
asslqsgvps rvsgtgsgtd ftltisslqp edfatyycqq syspsftfgp 100
gtkvdIK 107
Protein Sequence Defining the Kappa Chain Variable Region of the
28042.7 Antibody
(SEQ ID NO: 29)
diqmtqspss lsasvgdrvt itcrasqsis Sylnwyqqkp gkapklliyA 50
asslqsgvps rvsgtgsgtd ftltisslqp edfatyycqq syspsftfgp 100
gtkvdIK 107
Protein Sequence Defining the Kappa Chain Variable Region of the
28042.8 Antibody
(SEQ ID NO: 37)
diqmtqspss lsasvgdrvt itcrasqsis nylnwyqqkp gkapklliyg 50
asslqsgvps rFsgtgsgtd ftltisslqp edfatyycqq syspsftfgp 100
gtkvdfe 107
Protein Sequence Defining the Kappa Chain Variable Region of the
28042.9 Antibody
(SEQ ID NO: 38)
diqmtqspss lsasvgdrvt itcrasqsis nylnwyqqkp gkapklliyg 50
asslqsgvps rvsgSgsgtd ftltisslqp edfatyycqq syspsftfgp 100
gtkvdfe 107
Protein Sequence Defining the Kappa Chain Variable Region of the
28042.10 Antibody
(SEQ ID NO: 39)
diqmtqspss lsasvgdrvt itcrasqsis Sylnwyqqkp gkapklliyA 50
asslqsgvps rFsgSgsgtd ftltisslqp edfatyycqq syspsftfgp 100
gtkvdfe 107
Protein Sequence Defining the Kappa Chain of the 28042.1 Antibody
(SEQ ID NO: 30)
diqmtqspss lsasvgdrvt itcrasqsis Sylnwyqqkp gkapklliyg 50
asslqsgvps rvsgtgsgtd ftltisslqp edfatyycqq syspsftfgp 100
gtkvdfertv aapsvfifpp sdeqlksgta svvellnnfy preakvqwkv 150
dnalqsgnsq esvteqdskd styslsstlt lskadyekhk vyacevthqg 200
lsspvtksfn rgec 214
Protein Sequence Defining the Kappa Chain of the 28042.2 Antibody
(SEQ ID NO: 31)
digmtgspss lsasvgdrvt itcrasqsis nylnwyqqkp gkapklliyA 50
asslqsgvps rvsgtgsgtd ftltisslqp edfatyycqq syspsftfgp 100
gtkvdfertv aapsvfifpp sdeqlksgta svvellnnfy preakvqwkv 150
dnalqsgnsq esvteqdskd styslsstlt lskadyekhk vyacevthqg 200
lsspvtksfn rgec 214
10
Protein Sequence Defining the Kappa Chain of the 28042.3 Antibody
(SEQ ID NO: 32)
digmtgspss lsasvgdrvt itcrasqsis nylnwyqqkp gkapklliyg 50
asslqsgvps rvsgtgsgtd ftltisslqp edfatyycqq syspsftfgp 100
gtkvdIertv aapsvfifpp sdeqlksgta svvellnnfy preakvqwkv 150
dnalqsgnsq esvteqdskd styslsstlt lskadyekhk vyacevthqg 200
lsspvtksfn rgec 214
Protein Sequence Defining the Kappa Chain of the 28042.4 Antibody
(SEQ ID NO: 33)
digmtgspss lsasvgdrvt itcrasqsis nylnwyqqkp gkapklliyg 50
asslqsgvps rvsgtgsgtd ftltisslqp edfatyycqq syspsftfgp 100
gtkvdfKrtv aapsvfifpp sdeqlksgta svvellnnfy preakvqwkv 150
dnalqsgnsq esvteqdskd styslsstlt lskadyekhk vyacevthqg 200
lsspvtksfn rgec 214
Protein Sequence Defining the Kappa Chain of the 28042.5 Antibody
(SEQ ID NO: 34)
digmtgspss lsasvgdrvt itcrasqsis Sylnwyqqkp gkapklliyA 50
asslqsgvps rvsgtgsgtd ftltisslqp edfatyycqq syspsftfgp 100
gtkvdfertv aapsvfifpp sdeqlksgta svvellnnfy preakvqwkv 150
dnalqsgnsq esvteqdskd styslsstlt lskadyekhk vyacevthqg 200
lsspvtksfn rgec 214
Protein Sequence Defining the Kappa Chain of the 28042.6 Antibody
(SEQ ID NO: 35)
digmtgspss lsasvgdrvt itcrasqsis nylnwyqqkp gkapklliyg 50
asslqsgvps rvsgtgsgtd ftltisslqp edfatyycqq syspsftfgp 100
gtkvdIKrtv aapsvfifpp sdeqlksgta svvellnnfy preakvqwkv 150
dnalqsgnsq esvteqdskd styslsstlt lskadyekhk vyacevthqg 200
lsspvtksfn rgec 214
Protein Sequence Defining the Kappa Chain of the 28042.7 Antibody
(SEQ ID NO: 36)
digmtgspss lsasvgdrvt itcrasqsis Sylnwyqqkp gkapklliyA 50
asslqsgvps rvsgtgsgtd ftltisslqp edfatyycqq syspsftfgp 100
gtkvdIKrtv aapsvfifpp sdeqlksgta svvellnnfy preakvqwkv 150
dnalqsgnsq esvtegdskd styslsstlt lskadyekhk vyacevthqg 200
lsspvtksfn rgec 214
Protein Sequence Defining the Kappa Chain of the 28042.8 Antibody
(SEQ ID NO: 40)
digmtgspss lsasvgdrvt itcrasqsis nylnwyqqkp gkapklliyg 50
asslqsgvps rFsgtgsgtd ftltisslqp edfatyycqq syspsftfgp 100
gtkvdfertv aapsvfifpp sdeqlksgta svvellnnfy preakvqwkv 150
dnalqsgnsq esvtegdskd styslsstlt lskadyekhk vyacevthqg 200
lsspvtksfn rgec 214
Protein Sequence Defining the Kappa Chain of the 28042.9 Antibody
(SEQ ID NO: 41)
digmtgspss lsasvgdrvt itcrasqsis nylnwyqqkp gkapklliyg 50
asslqsgvps rvsgSgsgtd ftltisslqp edfatyycqq syspsftfgp 100
gtkvdfertv aapsvfifpp sdeqlksgta svvellnnfy preakvqwkv 150
dnalqsgnsq esvtegdskd styslsstlt lskadyekhk vyacevthqg 200
lsspvtksfn rgec 214
Protein Sequence Defining the Kappa Chain of the 28042.10
Antibody
(SEQ ID NO: 42)
digmtgspss lsasvgdrvt itcrasqsis Sylnwyqqkp gkapklliyA 50
asslqsgvps rFsgSgsgtd ftltisslqp edfatyycqq syspsftfgp 100
gtkvdfertv aapsvfifpp sdeqlksgta svvellnnfy preakvqwkv 150
dnalqsgnsq esvtegdskd styslsstlt lskadyekhk vyacevthqg 200
lsspvtksfn rgec 214

Table 5 provides the heavy chain and light chain full length sequences for the 28042 antibody and antibody variants 28042.1-28042.10.

TABLE 5

| | | Light Chain |
|---|---|---|
| Antibody Name | Heavy Chain Protein Sequence | Protein Sequence |
| 28042 | SEQ ID NO: 1 | SEQ ID NO: 2 |
| 28042.1 | SEQ ID NO: 1 | SEQ ID NO: 30 |
| 28042.2 | SEQ ID NO: 1 | SEQ ID NO: 31 |
| 28042.3 | SEQ ID NO: 1 | SEQ ID NO: 32 |
| 28042.4 | SEQ ID NO: 1 | SEQ ID NO: 33 |
| 28042.5 | SEQ ID NO: 1 | SEQ ID NO: 34 |
| 28042.6 | SEQ ID NO: 1 | SEQ ID NO: 35 |
| 28042.7 | SEQ ID NO: 1 | SEQ ID NO: 36 |
| 28042.8 | SEQ ID NO: 1 | SEQ ID NO: 40 |
| 28042.9 | SEQ ID NO: 1 | SEQ ID NO: 41 |
| 28042.10 | SEQ ID NO: 1 | SEQ ID NO: 42 |

Table 6 provides combinations of heavy chain variable region protein sequences, heavy chain constant region sequences, light chain variable region sequences and light chain constant region sequences for the 28042 antibody and antibody variants 28042.1-28042.10.

TABLE 6

| | Heavy Chain | Heavy Chain | Light Chain | Light Chain |
|---|---|---|---|---|
| Antibody | Variable Protein | Constant Region | Variable Region | Constant Region |
| Name | Sequence | Sequence | Sequence | Sequence |
| 28042 | SEQ ID NO: 6 | SEQ ID NO: 13 | SEQ ID NO: 10 | SEQ ID NO: 14 |
| 28042.1 | SEQ ID NO: 6 | SEQ ID NO: 13 | SEQ ID NO: 23 | SEQ ID NO: 14 |
| 28042.2 | SEQ ID NO: 6 | SEQ ID NO: 13 | SEQ ID NO: 24 | SEQ ID NO: 14 |
| 28042.3 | SEQ ID NO: 6 | SEQ ID NO: 13 | SEQ ID NO: 25 | SEQ ID NO: 14 |
| 28042.4 | SEQ ID NO: 6 | SEQ ID NO: 13 | SEQ ID NO: 26 | SEQ ID NO: 14 |
| 28042.5 | SEQ ID NO: 6 | SEQ ID NO: 13 | SEQ ID NO: 27 | SEQ ID NO: 14 |
| 28042.6 | SEQ ID NO: 6 | SEQ ID NO: 13 | SEQ ID NO: 28 | SEQ ID NO: 14 |
| 28042.7 | SEQ ID NO: 6 | SEQ ID NO: 13 | SEQ ID NO: 29 | SEQ ID NO: 14 |
| 28042.8 | SEQ ID NO: 6 | SEQ ID NO: 13 | SEQ ID NO: 37 | SEQ ID NO: 14 |
| 28042.9 | SEQ ID NO: 6 | SEQ ID NO: 13 | SEQ ID NO: 38 | SEQ ID NO: 14 |
| 28042.10 | SEQ ID NO: 6 | SEQ ID NO: 13 | SEQ ID NO: 39 | SEQ ID NO: 14 |

Table 7 discloses the CDR sequences for antibody 28042 and variants 28042.1-28042.10.

TABLE 7

| Antibody | CDR_{H1} | CDR_{H2} | CDR_{H3} | CDR_{L1} | CDR_{L2} | CDR_{L3} |
|---|---|---|---|---|---|---|
| 28042 | SEQ ID | SEQ ID | SEQ ID | SEQ ID | SEQ ID | SEQ ID |
| | NO: 3 | NO: 4 | NO: 5 | NO: 7 | NO: 8 | NO: 9 |
| 28042.1 | SEQ ID | SEQ ID | SEQ ID | SEQ ID | SEQ ID | SEQ ID |
| | NO: 3 | NO: 4 | NO: 5 | NO: 21 | NO: 8 | NO: 9 |
| 28042.2 | SEQ ID | SEQ ID | SEQ ID | SEQ ID | SEQ ID | SEQ ID |
| | NO: 3 | NO: 4 | NO: 5 | NO: 7 | NO: 22 | NO: 9 |
| 28042.3 | SEQ ID | SEQ ID | SEQ ID | SEQ ID | SEQ ID | SEQ ID |
| | NO: 3 | NO: 4 | NO: 5 | NO: 7 | NO: 8 | NO: 9 |
| 28042.4 | SEQ ID | SEQ ID | SEQ ID | SEQ ID | SEQ ID | SEQ ID |
| | NO: 3 | NO: 4 | NO: 5 | NO: 7 | NO: 8 | NO: 9 |
| 28042.5 | SEQ ID | SEQ ID | SEQ ID | SEQ ID | SEQ ID | SEQ ID |
| | NO: 3 | NO: 4 | NO: 5 | NO: 21 | NO: 22 | NO: 9 |
| 28042.6 | SEQ ID | SEQ ID | SEQ ID | SEQ ID | SEQ ID | SEQ ID |
| | NO: 3 | NO: 4 | NO: 5 | NO: 7 | NO: 8 | NO: 9 |
| 28042.7 | SEQ ID | SEQ ID | SEQ ID | SEQ ID | SEQ ID | SEQ ID |
| | NO: 3 | NO: 4 | NO: 5 | NO: 21 | NO: 22 | NO: 9 |
| 28042.8 | SEQ ID | SEQ ID | SEQ ID | SEQ ID | SEQ ID | SEQ ID |
| | NO: 3 | NO: 4 | NO: 5 | NO: 7 | NO: 8 | NO: 9 |
| 28042.9 | SEQ ID | SEQ ID | SEQ ID | SEQ ID | SEQ ID | SEQ ID |
| | NO: 3 | NO: 4 | NO: 5 | NO: 7 | NO: 8 | NO: 9 |
| 28042.10 | SEQ ID | SEQ ID | SEQ ID | SEQ ID | SEQ ID | SEQ ID |
| | NO: 3 | NO: 4 | NO: 5 | NO: 21 | NO: 22 | NO: 9 |

### Example 2: Binding Affinities

The binding affinity and kinetics of binding of the 28042 antibody to recombinant human Notch3 extracellular domain (containing EGF like domains 1-11) Fc fusion protein (rhNotch3-Fc (R&D Systems, Inc., Minneapolis, MN)) can be measured by surface plasmon resonance using a Biacore® T100 instrument (GE Healthcare, Piscataway, NJ).

Goat anti-human IgG is immobilized on carboxymethylated dextran CM4 sensor chips (GE Healthcare) by amine coupling, according to a standard protocol. Analyses are performed at 25° C. and 37° C. using PBS containing 0.05% surfactant P20 as running buffer. The antibody is captured in individual flow cells at a flow rate of 10 μl/minute. Injection time is varied for each antibody to yield an Rmax between 30 and 60 resonance units (RU). 250 μg/mL human IgG Fc is injected to block non-specific binding of Fc (from the Notch3 fusion protein) to the capture antibody. Buffer or rhNotch3-Fc diluted in running buffer is injected sequentially over a reference surface (no antibody captured) and the active surface (antibody to be tested) for 240 seconds at 60 μL/minute. The dissociation phase is monitored for up to 1500 seconds. The surface is then regenerated with two 60-second injections of 10 mM Glycine-HCl, pH 2.25, at a flow rate of 30 μL/minute. The rhNotch3-Fc concentration range tested is 100 nM to 3.125 nM (2 fold dilution).

Kinetic parameters can be determined using the kinetic function of the BIAevaluation software (GE Healthcare) with double reference subtraction. Kinetic parameters for the antibody, kₐ (association rate constant), k_{d} (dissociation rate constant) and K_{D} (equilibrium dissociation constant) are determined.

Kinetic values of the monoclonal antibody 28042 on rhNotch3-Fc at 25° C. and 37° C. are summarized in Table 8.

TABLE 8
Antibody Binding to rhNotch3-Fc

| Temperature | kₐ (1/Ms) | k_{d} (1/s) | K_{D} (M) |
|---|---|---|---|
| 25° C. | 1.77E+05 | 2.99E−04 | 2.55E−09 |
| 37° C. | 9.47E+04 | 3.96E−04 | 4.18E−09 |

The data in Table 8 demonstrate that the 28042 antibody binds rhNotch3-Fc with a K_{D} of about 1-10 nM, 10 nM, 9 nM, 8 nM, 7 nM, 6 nM, 5 nM, 4 nM, 3 nM, 2 nM, 1 nM, 900 pM, 850 pM, 800 pM, 750 pM, 700 pM, 650 pM, 600 pM, 500 pM, 400 pM, 300 pM, or 200 pM or less.

Binding to cell surface human Notch3 by the antibody 28042 can be measured at 4° C., using Fluorescence Activated Cell Sorting (FACS). CHO N3 (Flp-In-CHO cells (Invitrogen) stably transfected with human Notch3), HCC2429 cells, and RL-952 cells expressing human Notch3 are washed once with PBS containing calcium chloride and magnesium chloride (Invitrogen) and harvested using cell dissociation buffer (Invitrogen). Cells are washed a second time with PBS and resuspended in FACS buffer (PBS with 0.5% BSA (Sigma-Aldrich)) for a final cell concentration of 250,000 cells per well into a 96-well v-bottom plate. Purified antibodies are diluted in FACS buffer over a concentration range of 100 nM to 0 nM. Cells are then incubated at 4° C. with 100 μL of antibody for one hour, washed with FACS buffer twice, and resuspended in 100 μL of goat-anti mouse PE-conjugated antibody (Jackson Immuno Research). Cells are incubated at 4° C. for 30 minutes in the dark, washed once with FACS buffer, and then analyzed using a Beckman Coulter Cytomics FC 500 instrument. The geometric mean of the florescent intensity then is calculated for each antibody concentration. These values then are entered into Prism software (GraphPad, La Jolla, CA) and used to generate a binding curve by plotting geometric mean versus antibody concentration. From the binding curve, the following equation is used to calculate the K_{D} and K_{D} range of 28042 antibody binding to human Notch3 on the cell surface of the three cell lines.

$$One site binding ( hyperbola ) \qquad Equation$$

$$Y = B max * X / ( {K}_{D} + X )$$

* describes the binding of a ligand to a receptor that follows the law of mass action. Bmax is the maximal binding, and K_{D} is the concentration of ligand required to reach half-maximal binding.

The results for the 28042 antibody are summarized in Table 9.

TABLE 9
Binding to hNotch3 on Cell Surface

| K_{D} (nM) | K_{D} Range (nM) |
|---|---|
| 2.723 | 1.794 to 3.652 |

The data in Table 9 demonstrate that the 28042 antibody binds rhNotch3-Fc with a K_{D} of about 1-10 nM, 10 nM, 9 nM, 8 nM, 7 nM, 6 nM, 5 nM, 4 nM, 3 nM, 2 nM, 1 nM, 900 pM, 850 pM, 800 pM, 750 pM, 700 pM, 650 pM, 600 pM, 500 pM, 400 pM, 300 pM, or 200 pM or less.

K_{D} values for the binding of the 28042 and 28042.1-28042.7 antibodies to cynoNotch3 was tested according to a similar protocol as described above for 28042 antibody binding to hNotch3, and the results are shown in Table 10.

TABLE 10
Binding to cynoNotch3 monomer at 25° C.

| Antibody | K_{D} (M) |
|---|---|
| 28042.1 | 5.35E−09 |
| 28042.2 | 2.14E−07 |
| 28042.3 | 2.65E−07 |
| 28042.4 | 3.36E−07 |
| 28042.5 | 2.11E−07 |
| 28042.6 | 1.92E−07 |
| 28042.7 | 2.51E−07 |
| 28042 | 8.06E−07 |

### Example 3: Binding Specificity

In this example, the binding specificity of the 28042 antibody for human Notch1, human Notch2, human Notch3, or murine Notch3 proteins is described.

The specificity of the 28042 antibody binding for Notch-Fc fusion proteins can be determined using surface plasmon resonance using a Biacore® T100 instrument (GE Healthcare, Piscataway, NJ). Rabbit anti-mouse IgGs (GE Healthcare) are immobilized on carboxymethylated dextran CM4 sensor chips (GE Healthcare) by amine coupling, according to a standard protocol. Analyses are performed at 25° C. and 37° C. using PBS containing 0.05% surfactant P20 as running buffer. The antibody is captured in individual flow cells at a flow rate of 10 μL/minute. Injection time is varied for each antibody to yield an Rmax between 30 and 60 RU. 250 μg/mL human IgG Fc is injected to block non-specific binding of Fc (from the Notch3 fusion protein) to the capture antibody. Buffer or rhNotch1-Fc (R&D Systems, Minneapolis, MN; Cat. No. 3647-TK-050), rhNotch2-Fc (R&D Cat. No. 3735-NT-050), and rhNotch3-Fc (R&D Cat. No. 1559-NT-050), diluted in running buffer is injected sequentially over a reference surface (no antibody captured) and the active surface (antibody to be tested) for 240 seconds at 60 μL/minute. The dissociation phase is monitored for up to 1500 seconds. The surface is then regenerated with two 60-second injections of 10 mM Glycine-HCl, pH 2.25, at a flow rate of 30 μL/minute. The rhNotch3-Fc, rhNotch2-Fc, and rhNotch1-Fc concentration range tested are 100 nM to 3.125 nM (2 fold dilution).

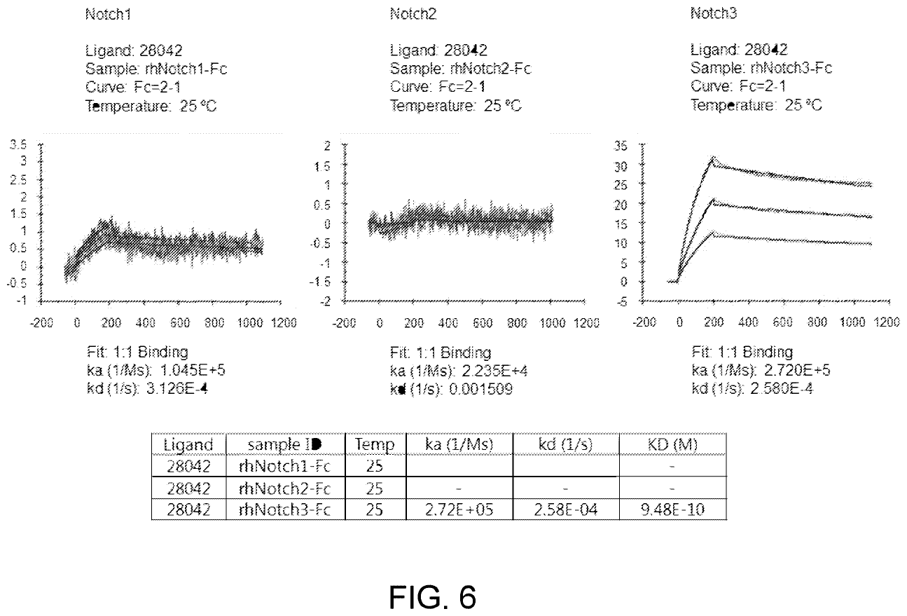

As shown in FIG. 6, the specificity of the 28042 antibody binding to the extracellular domains of Notch1-Fe, Notch2-Fc, and Notch3-Fc fusion proteins was measured at three different dilutions by surface plasmon resonance. As shown in FIG. 6, the binding affinity of the 28042 antibody for Notch3 is high while there is no appreciable binding of the 28042 antibody to Notch1 or Notch2.

To determine the specificity of binding to cell surface Notch proteins, stable cells lines expressing Notch receptors are produced by transfecting FlpIn™ CHO or FlpIn™ 293 cells (Life Technologies, Grand Island, NY) with full length human Notch1, Notch2, Notch3, or full length murine Notch3 cDNAs cloned into the pcDNA5FRT vector using Lipofectamine 2000 (Life Technologies) according to the manufacturer's protocol. Twenty-four hours after transfection, CHO cells are split into F12 media containing 10% FBS, 2 mM L-Glutamine and 600-700 μg/ml hygromycin B (Sigma-Aldrich, St. Louis, MO) to select for transfected cells. 293 cells are split into DMEM media containing 10% FBS, 2 mM L-glutamine and 200 μg/ml hygromycin B. Expression of Notch receptors can be confirmed by FACS analysis using anti-human Notch1 PE (BioLegend, San Diego, CA), anti-Notch2 PE (eBioscience, San Diego, CA), or anti-human Notch3 PE (BioLegend, San Diego, CA).

To determine the specificity of binding to cell surface Notch proteins, the 28042 antibody is tested for binding to human Notch1, human Notch2, human Notch3 and murine Notch3 expressed on the surface of CHO cells by FACS analysis. Full length Notch receptor cDNAs cloned into pcDNA5FRT were transfected into FlpIn™ CHO cells and selected with 600 μg/ml hygromycin (Sigma), according to the manufacturer's protocol. Cell surface expression of Notch receptors is confirmed by FACS analysis using anti-Notch1 PE, anti-Notch2 PE or anti-Notch3 PE control antibodies (Biolegend). FlpIn™ CHO cells expressing Notch receptors are incubated with 5 μg/ml 28042 antibody or human IgG control antibody (Jackson ImmunoResearch) for 1 hour on ice, followed by washing with PBS/0.5% BSA, and incubation with anti-human PE secondary antibody (Biolegend) for 30 minutes on ice. FACS is performed on a BD FACSCanto II cell analyzer (BD Biosciences) and analyzed using FlowJo software.

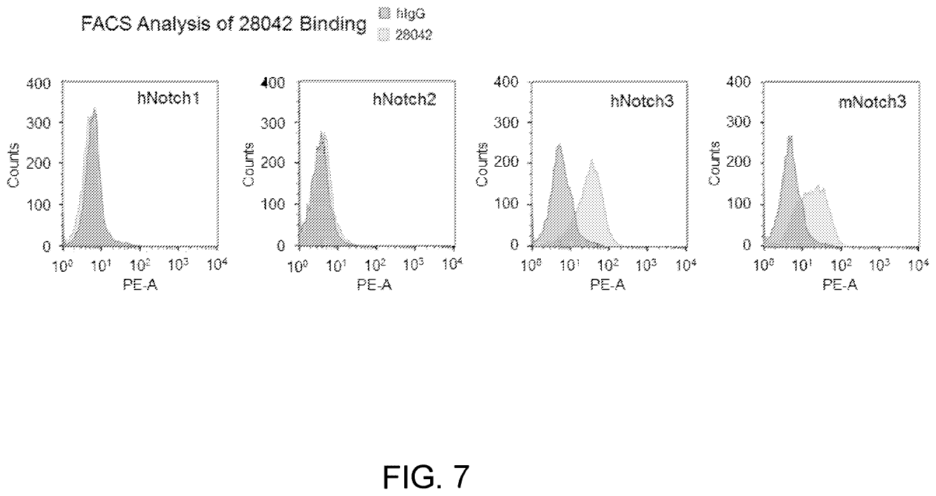

As shown in FIG. 7, FACS binding data demonstrates that the 28042 antibody specifically binds to human Notch3 and murine Notch3, with no cross reactivity with human Notch1 or human Notch2.

To determine the specificity of binding to cell surface Notch proteins, the 28042 antibody is tested for binding to human Notch1, human Notch2, human Notch3 and murine Notch3 expressed on the surface of CHO cells using electrochemiluminescence (Meso Scale Discovery). A CHO line lacking any human Notch protein is used as a negative control. Cells are grown under standard conditions (37° C., F12+10% FBS). For binding studies, cells are washed in PBS containing calcium and magnesium, and removed from the plate by treatment with Cell Dissociation Buffer (Life Technologies) for ten minutes at 37° C.

Cells are seeded at a density of 30,000 cells per well, in hybridoma media, in a standard 96-well binding plates (Meso Scale Discovery, Cat. No. L15XA-6). Cells are incubated for one hour at 37° C. Antibodies or control IgG are added at 5 μg/mL, in 50 μL hybridoma media, and incubated for one hour at 37° C. The plates are washed twice with PBS containing 3% BSA. Binding of the antibodies to cell surface is detected using 2 μg/mL of MSD anti-mouse IgG secondary antibody (Meso Scale Discovery, Cat. No. R32AC-1) for one hour at 4° C. Plates are washed twice with PBS containing 3% BSA, and 150 μL of read buffer (Meso Scale Discovery Cat. No. R92TC-1) is added. The plates are analyzed on a Sector Imager 2400 instrument (Meso Scale Discovery).

### Examples 4: Inhibition of Notch3-Ligand Binding

The 28042 antibody can be tested for its ability to inhibit the binding of rhNotch3 binding to human Jag1, Jag2, DLL1 and DLL4. Binding measurements are made by bio-layer interferometry (BLI), using a FortéBio Octet® QK instrument (FortéBio, Menlo Park, CA). The ligands tested are rhJag1-Fc (R&D Cat. No. 1277-JG-050), rhJag2-Fc (R&D Cat. No. 1726-JG-050), rhDLL1-Fc (R&D Cat. No. 5026-DL-050), and His tagged rhDLL4 (R&D Cat. No. 1506-D4-050).

To determine the degree of inhibition of Notch3-ligand binding by the 28042 antibody, the Octet sensors are loaded with recombinant human Notch3, and the antibody is allowed to bind. Then sensors are immersed in 500 μg/mL human IgG to block non-specific binding. Ligands are prepared at a concentration of 400 nM in PBS containing 3% BSA, and are allowed to bind. The on-rate and off-rate for ligand binding are detected using the Octet QK instrument and software.

### Example 5: Inhibition of Ligand-Induced Notch3 ICD Cleavage

Activation of Notch receptors by ligands results in cleavage of the Notch intracellular domain (NICD), which can be detected by Western blot. In this example, the 28042 antibody is tested for its ability to inhibit ligand-induced cleavage of the Notch3 ICD.

To create soluble Notch ligands, PCR is used to amplify sequences corresponding to the extracellular domains of human Jag1 or human Jag2 cDNA and fuse them in-frame to the coding sequence of human or murine IgG Fc. This construct then is subcloned into the pEE14.4 expression vector (Lonza), transfected into CHOK1SV cells, and selected to produce stable cell lines that secrete hJag1-hFc, hJag1-mFc, hJag2-hFc, or hJag2-mFc fusion protein. Fusion proteins are purified from cell supernatants.

In one experiment, mNotch3 expressing cells are incubated with the Notch3 ligand Jag1, and ligand-induced cleavage of the mNotch3 ICD is measured by Western blot. The 28042 antibody is further added to test for its ability to inhibit ligand-induced cleavage of the mNotch3 ICD. 96-well Immunosorp ELISA plates (Nalgene Nunc, Rochester, NY) are coated with 5 μg/ml anti-mouse Fc (Jackson ImmunoResearch, West Grove, PA) overnight at 4° C. After washing wells with PBS/0.5% BSA, 5 μg/ml of soluble hJag1-mFc fusion protein is added and allowed to bind at room temperature for two hours. Unbound protein is removed by washing with PBS/0.5% BSA. 293 FlpIn™ cells engineered to express mNotch3 (produced as described in Example 3) are plated on captured ligand or mFc in the presence of 10 μg/mL human IgG control, the 28042 antibody, the Notch 3 antibody ABX, or an antibody specific for human Notch 3 (04F11) Cells are lysed 24 hours later in RIPA buffer (Boston BioProducts, Ashland, MA) containing protease inhibitors. Lysates are run on SDS PAGE, and transferred to nitrocellulose. Induction of NICD cleavage can be detected by probing with an antibody against the Notch3 C-terminus (Cell Signaling, Danvers, MA) that detects both full length protein and the cleaved ICD.

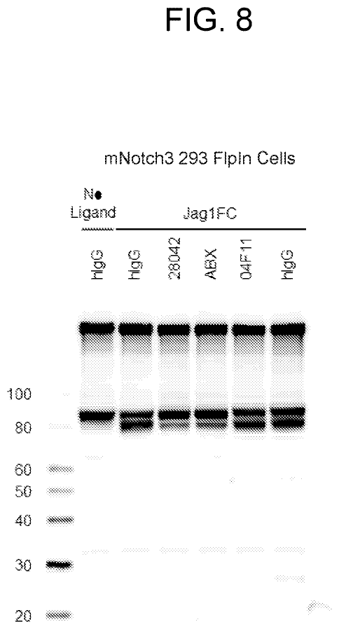

As shown in FIG. 8, plating mNotch3 293 FlpIn™ cells on Jag1-mFc ligand induces ICD cleavage and activation of the receptor. Addition of the 28042 antibody diminishes ligand-induced cleavage and activation of mNotch3, while the addition of a human IgG control or a human Notch3 control antibody has no effect on mNotch3 ICD cleavage.

In another experiment, Notch3 expressing cells are incubated with the Notch3 ligand DLL4, and ligand-induced cleavage of the Notch3 ICD is measured by Western blot. The 28042 antibody is further added to test for its ability to inhibit ligand-induced cleavage of the Notch3 ICD. 96-well Immunosorp ELISA plates are coated with 5 μg/mL His tag antibody (R&D Systems, Minneapolis, MN) overnight at 4° C., after which 5 μg/mL DLL4-His fusion protein (R&D Systems, Minneapolis, MN), is added and allowed to bind for two hours at room temperature. Unbound protein is removed by washing with PBS/0.5% BSA. NCI-H838 cells are plated on captured ligand in the presence of 10 μg/mL human IgG control, the 28042 antibody, the Notch 3 antibody ABX, or an antibody specific for human Notch 3 (04F11). Cells are lysed 24 hours later in RIPA buffer (Boston BioProducts, Ashland, MA) containing protease inhibitors. Lysates are run on SDS PAGE, and transferred to nitrocellulose. Induction of NICD cleavage can be detected by probing with an antibody against the Notch3 C-terminus (Cell Signaling, Danvers, MA) that detects both full length protein and the cleaved ICD.

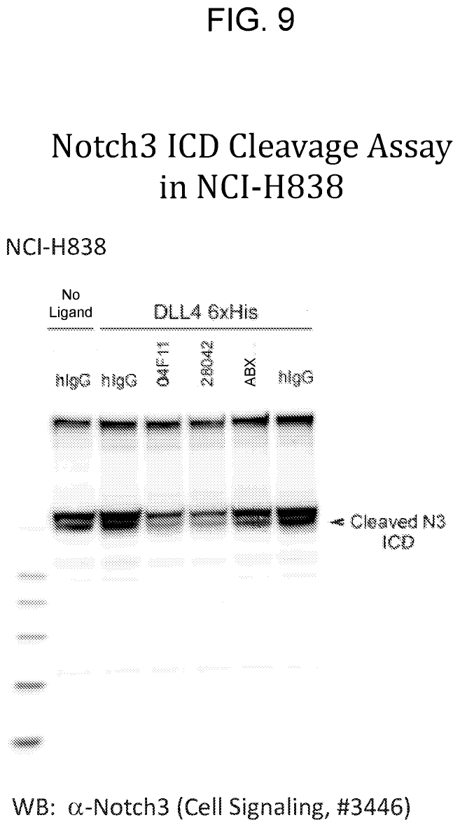

NCI-H838 cells exhibit low levels of constitutive human Notch3 activation as evidenced by the presence of cleaved N3 ICD even in absence of exogenous ligand, as shown in the Western blot in FIG. 9. As shown in FIG. 9, the 28042 antibody blocks further cleavage and activation of the Notch3 receptor in response to DLL4 stimulation and reduces the level of cleaved Notch3 ICD to below baseline levels, while a human IgG control antibody had no effect on Notch3 cleavage and activation.

In another experiment, murine or human Notch3 expressing cells are incubated with the Notch3 ligands Jag1, Jag2, and DLL4, and ligand-induced cleavage of the Notch3 ICD is measured by Western blot. The 28042 antibody is further added to test for its ability to inhibit ligand-induced cleavage of the Notch3 ICD. 96-well Maxisorp plates (Nunc) are coated overnight with 5pg/ml anti-His tag (R&D Systems) or anti-mFc (Jackson ImmunoResearch) antibodies. After washing, 5pg/ml recombinant DLL4 (R&D Systems, Minneapolis, MN), hJag1-mFc, or hJag2-mFc in PBS/0.5% BSA are added and allowed to bind for two hours at room temperature. No ligand control wells are coated with anti-mFc antibody overnight and incubated with 5pg/ml mFc (Jackson ImmunoResearch). FlpIn™ CHO cells stably expressing human Notch3 or murine Notch3 are pre-incubated with 10 μg/ml of the 28042 antibody or hIgG control antibody for 30 minutes before plating 30,000 cells per well onto ligand-coated plates and incubating at 37° C. overnight. Cells are lysed 24 hours later in RIPA buffer (Boston BioProducts, Ashland, MA) containing protease inhibitors. Lysates are run on SDS PAGE, and transferred to nitrocellulose. Induction of NICD cleavage can be detected by probing with an antibody against the Notch3 C-terminus (Cell Signaling, Danvers, MA) that detects both full length protein and the cleaved ICD. An antibody against GAPDH (Cell Signaling) is used as a loading control. Bands are detected using an Odyssey CLx imaging system and Image Studio software (LI-COR).

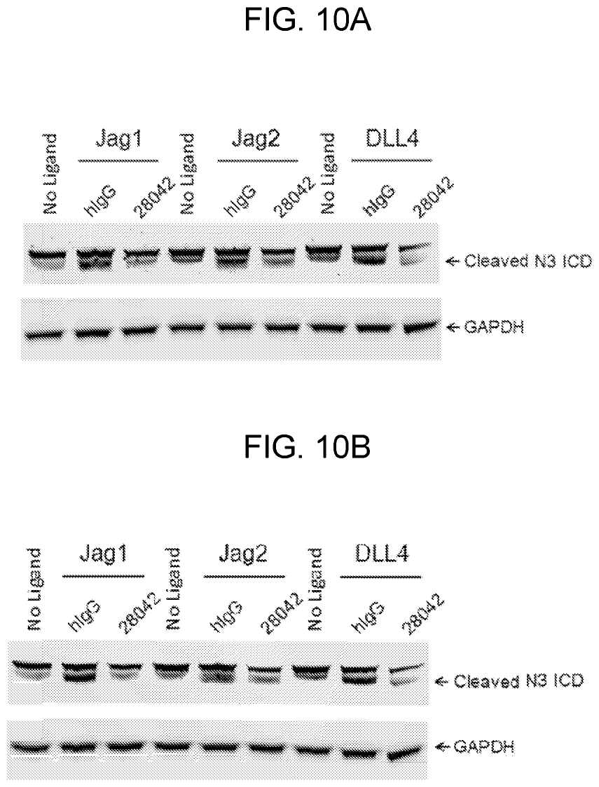

As shown in FIG. 10, in mouse (FIG. 10A) and human (FIG. 10B) Notch3-expressing cells, the 28042 antibody blocks Jag1, Jag2 or DLL4 ligand-induced cleavage and activation of Notch3.

Collectively, these results demonstrate the ability of the 28042 antibody to inhibit ligand-induced activation of Notch3.

### Example 6: Inhibition of Notch3-Dependent Transcription

In this example, the 28042 antibody is tested for its ability to inhibit the transcription of Notch3 target genes. Reporter cells are generated that expressed luciferase in response to Notch3 activation, and the resulting luminescence is measured in the presence or absence of the 28042 antibody.

Reporter cell lines dependent upon Notch3 are produced via the lentiviral introduction of a RBP-Jκ-dependent luciferase reporter gene (SABiosciences, Frederick, MD) into 293-FlpIn Notch3 cells, RL95-2 endometrial cancer cells, HCC1143 breast cancer cells, and MDA-MB-468 breast cancer cells. To activate Notch3-dependent signaling and transcription, cells are plated on ligand-coated wells prepared as described in Example 5. Cells are pre-incubated with a 3-fold dilution series of Notch3 antibodies at concentrations ranging from 0-300 μg/mL, for one hour at 37° C., before seeding 100 μl of the suspension into 96-well plates coated with ligand or hFc. Cells are incubated in ligand-coated or murine-Fc-coated wells for four or twenty-four hours at 37° C., in 5% CO₂. Then, 100 μL of Promega Bright Glo™ (Promega, Madison, WI) is added to each well. The reaction is allowed to proceed for five minutes in the dark, and then the entire 200 p L volume is transferred into plates for analysis in a luminometer. Polyclonal antibodies directed against Notch1 (AF1057, R&D Systems), Notch2 (AF1190, R&D Systems) or Notch3 (AF1559, R&D Systems) are used as controls to confirm that ligand-stimulated reporter activity in each cell line is specifically dependent upon the introduced Notch receptor.

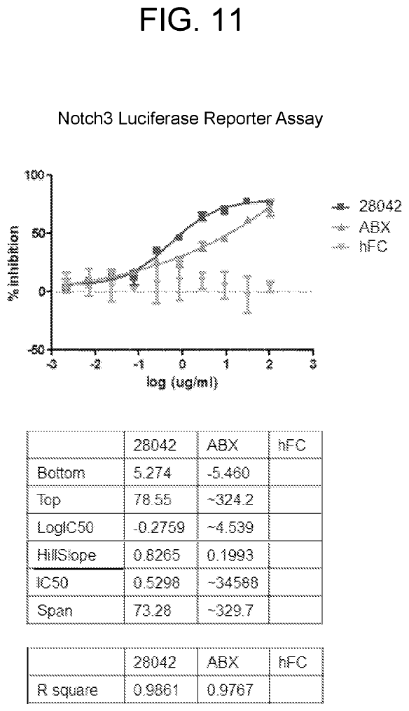

Data for a Notch3 Luciferase reporter assay for antibody 28042 is shown in FIG. 11. In this assay, cells of a MDA-MB-468 reporter cell line expressing endogenous human Notch3 and a RBP-Jκ luciferase reporter construct are plated on hJag2-mFc in the presence of Notch3 inhibitory antibodies. As shown therein, the 28042 antibody strongly inhibits the luminescent signal that results from Notch3 dependent transcription. The results demonstrate that the 28042 antibody inhibits Notch3-dependent transcription stimulated by the Jag2 ligand.

### Example 7: Inhibition of Tumor Growth

In this example, the 28042 antibody is tested for its ability to inhibit tumor growth in vivo in a genetically engineered Notch3-driven tumor model that overexpresses and is dependent on mNotch3 for tumor maintenance. Approximately eleven week old NCR nude mice (Taconic, Germantown, NY) are inoculated subcutaneously into the right flank with 2×10⁵ cells in 1:1 HBSS+Matrigel (Invitrogen, Carlsbad, CA)/Matrigel (BD Biosciences, San Jose, CA). Tumor measurements are taken twice weekly, using vernier calipers. Tumor volume is calculated using the formula: V=0.5×width×width×length. When tumors approach a volume of 150-200 mm³, mice are randomized into three groups of ten animals each. The next day, mice are treated with 20 mg/kg hIgG (control) or 20 mg/kg of the 28042 antibody or the ABX antibody by intraperitoneal injection. Mice are dosed twice weekly for the duration of the study. Forty-eight hours after final dosing, tumor volumes are measured again to assess tumor growth inhibition. All statistical analysis is done using GraphPad PRISM® Version 4.00. Final tumor volumes are analyzed using a one-way analysis of variance and Tukey multiple comparison test.

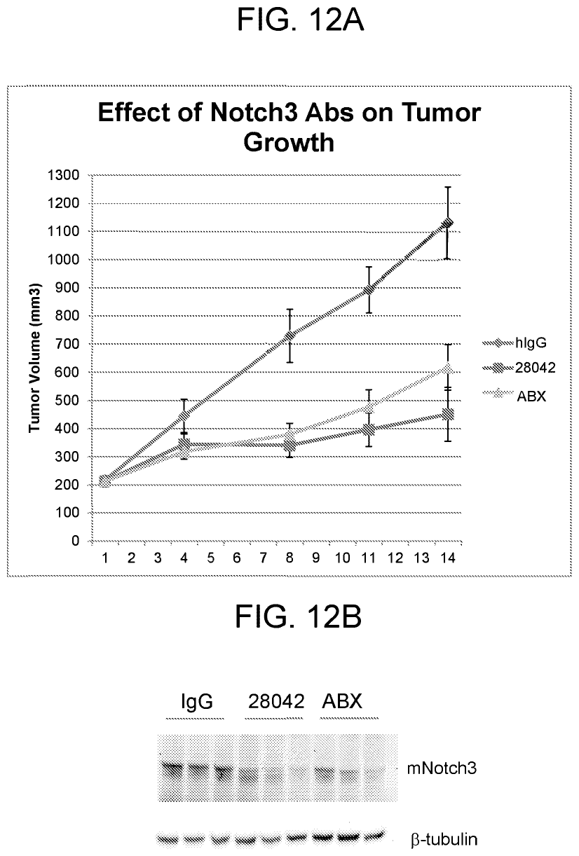

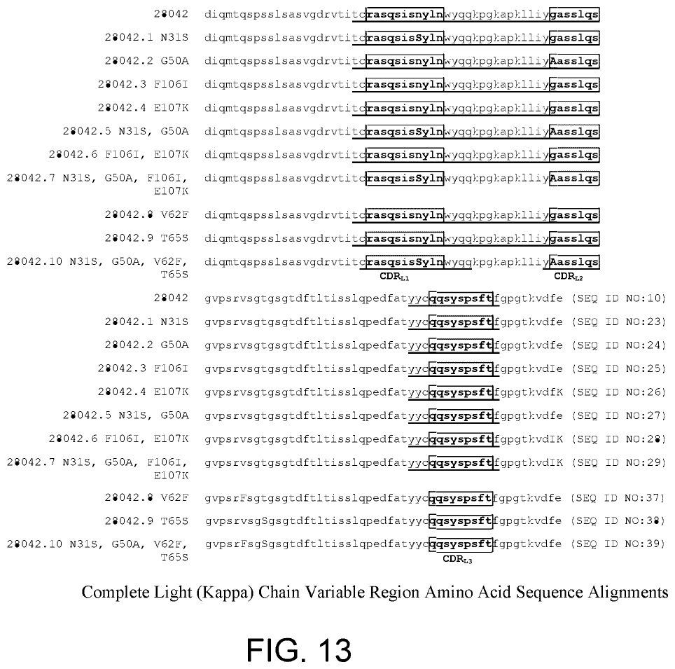

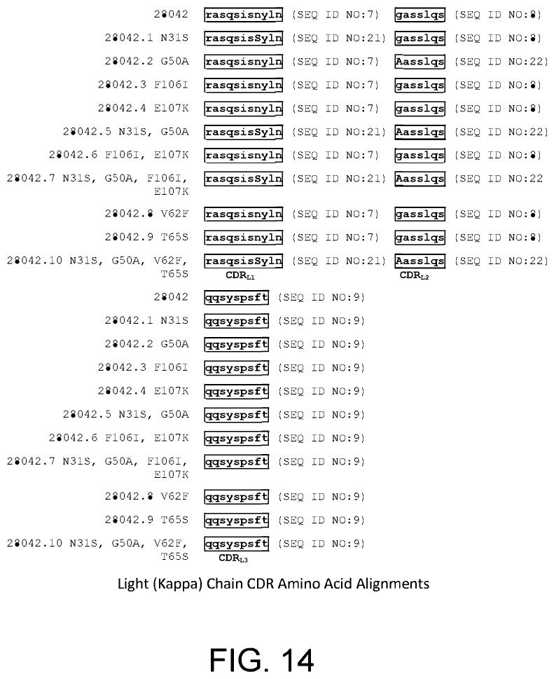

As shown in FIG. 12A, the treatment group (i.e., animals receiving the 28042 antibody) shows significant tumor growth inhibition, as compared to hIgG treated controls. All treatments are well-tolerated with no significant body weight loss.

As shown in FIG. 12B, lysates from harvested tumor cells treated with the 28042 antibody showed a significant decrease in human Notch3 ICD cleavage over those treated with the control antibody, showing that the 28042 antibody reduces Notch3 activation in tumor cells.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

1. An isolated antibody or a fragment thereof that specifically binds human Notch3, comprising:
(a) an immunoglobulin heavy chain variable region (VH) comprising a CDR_{H1}, CDR_{H2}, and CDR_{H3} of a VH amino acid sequence of SEQ ID NO: 6; and
(b) an immunoglobulin light chain variable region (VL) comprising a CDR_{L1}, CDR_{L2}, and CDR_{L3} of a VL comprising the amino acid sequence of SEQ ID NO: 10.

2. The isolated antibody or fragment thereof of claim 1, wherein the CDR_{H1}, CDR_{H2}, and CDR_{H3} comprise the amino acid sequences of SEQ ID NOs: 3, 4, and 5, respectively.

3. The isolated antibody or fragment thereof of claim 1, wherein the CDR_{L1}, CDR_{L2}, and CDR_{L3} comprise the amino acid sequences of SEQ ID NOs: 7, 8, and 9, respectively.

4. The isolated antibody or fragment thereof of claim 1, wherein the CDR_{H1}, CDR_{H2}, CDR_{H3}, CDR_{L1}, CDR_{L2}, and CDR_{L3} comprise the amino acid sequences of SEQ ID NOs: 3, 4, 5, 7, 8, and 9, respectively.

5. The isolated antibody or fragment thereof of claim 1, wherein the VH comprises an amino acid sequence at least 90% identical to the amino acid sequence of SEQ ID NO: 6.

6. The isolated antibody or fragment thereof of claim 1, wherein the VH comprises the amino acid sequence of SEQ ID NO: 6.

7. The isolated antibody or fragment thereof of claim 1, wherein the VL comprises an amino acid sequence at least 90% identical to the amino acid sequence of SEQ ID NO: 10.

8. The isolated antibody or fragment thereof of claim 1, wherein the VL comprises the amino acid sequence of SEQ ID NO: 10.

9. The isolated antibody or fragment thereof of claim 1, wherein the VH comprises an amino acid sequence at least 90% identical to the amino acid sequence of SEQ ID NO: 6 and the VL comprises an amino acid sequence at least 90% identical to the amino acid sequence of SEQ ID NO: 10.

10. The isolated antibody or fragment thereof of claim 1, wherein the VH comprises the amino acid sequence of SEQ ID NO: 6 and the VL comprises the amino acid sequence of SEQ ID NO: 10.

11. The isolated antibody or fragment thereof of claim 1, wherein the antibody comprises a heavy chain.

12. The isolated antibody or fragment thereof of claim 11, wherein the heavy chain comprises an amino acid sequence at least 90% identical to the amino acid sequence of SEQ ID NO: 1.

13. The isolated antibody or fragment thereof of claim 11, wherein the heavy chain comprises the amino acid sequence of SEQ ID NO: 1.

14. The isolated antibody or fragment thereof of claim 1, wherein the antibody comprises a light chain.

15. The isolated antibody or fragment thereof of claim 14, wherein the light chain comprises an amino acid sequence at least 90% identical to the amino acid sequence of SEQ ID NO: 2.

16. The isolated antibody or fragment thereof of claim 14, wherein the light chain comprises the amino acid sequence of SEQ ID NO: 2.

17. The isolated antibody or fragment thereof of claim 1, wherein the antibody comprises a heavy chain and a light chain.

18. The isolated antibody or fragment thereof of claim 17, wherein the heavy chain comprises an amino acid sequence at least 90% identical to the amino acid sequence of SEQ ID NO: 1 and the light chain comprises an amino acid sequence at least 90% identical to the amino acid sequence of SEQ ID NO: 2.

19. The isolated antibody or fragment thereof of claim 17, wherein the heavy chain comprises the amino acid sequence of SEQ ID NO: 1 and the light chain comprises the amino acid sequence of SEQ ID NO: 2.

20. The isolated antibody or fragment thereof of claim 17, comprising a first heavy chain, a second heavy chain, a first light chain, and a second light chain, wherein the first heavy chain and the second heavy chain comprise amino acid sequences at least 90% identical to the amino acid sequence of SEQ ID NO: 1 and the first light chain and the second light chain comprise amino acid sequences at least 90% identical to the amino acid sequence of SEQ ID NO: 2.

21. The isolated antibody or fragment thereof of claim 17, comprising a first heavy chain, a second heavy chain, a first light chain, and a second light chain, wherein the first heavy chain and the second heavy chain comprise the amino acid sequence of SEQ ID NO: 1 and the first light chain and the second light chain comprise the amino acid sequence of SEQ ID NO: 2.

22. A method of inhibiting or reducing ligand-induced Notch3 activity in a subject, the method comprising exposing the subject to an effective amount of the antibody of claim 1.

23. The method of claim 22, wherein the subject has a cancer selected from the group consisting of breast cancer, lung cancer, pancreatic cancer, endometrial cancer, ovarian cancer, prostate cancer, cervical cancer, brain cancer, skin cancer, colorectal cancer, gastric cancer, head and neck cancer, gastrointestinal cancer, sarcoma, and hematopoietic cell cancer.

24. The method of claim 22, wherein:
(a) the brain cancer is glioblastoma, astrocytoma, or neuroblastoma;
(b) the sarcoma is rhabdomyosarcoma or osteosarcoma; or
(c) the hematopoietic cell cancer is multiple myeloma or leukemia.

25. The method of claim 24, wherein the leukemia is T-ALL, B-ALL, or B-CLL.

26. The method of claim 22, wherein the ligand is selected from the group consisting of Jag1, Jag2, DLL1, and DLL2.

27. The method of claim 22, wherein the ligand-induced Notch3 activity is ligand-induced Notch3 intracellular domain (ICD) cleavage or ligand-induced Notch3-mediated transcription.

28. The method of claim 22, wherein the subject is a human.
